(19) United States
(12) Patent Application Publication
(10) Pub. No.: US 2026/0265675 A1
(43) Pub. Date: Sep. 10, 2026

(54) Methods for Enhanced Gene Expression in Microorganisms

(71) Applicant: ALGAE-C INC., Caledon (CA)

(72) Inventors: Natacha Mérindol, Trois-Rivieres (CA); Rémy Beauchemin, Trois-Rivieres (CA); Isabel Marie Anick Desgagné-penix, Trois-Rivieres (CA)

(21) Appl. No.: 19/167,086

(22) PCT Filed: Mar. 20, 2024
(86) PCT No.: PCT/CA2024/050332

§ 371 Date: Sep. 19, 2025

Related U.S. Application Data
(60) Provisional application No. 63/453,541, filed on Mar. 21, 2023.

(51) Int. Cl. C12N 1/125; C12N 9/99; C12N 15/67; C12R 1/89

(52) U.S. Cl. CPC C12N 1/125; C12N 9/99; C12N 15/67; C12R 2001/89

(57) ABSTRACT

The present invention relates to methods of increasing expression of an exogenous nucleic acid in a microorganism comprising adding a hydroxamate-based histone deacetylase inhibitor to a culture medium of the microorganism, wherein in some embodiments the hydroxamate-based deacetylase inhibitor has the structure of Formula (I): Formula (I) wherein R₁ is a cyclic or acyclic, substituted or unsubstituted, branched or unbranched aliphatic; a cyclic or acyclic, substituted or unsubstituted, branched or unbranched heteroaliphatic; a substituted or unsubstituted aryl, or a substituted or unsubstituted heteroaryl; and Formula (II) represents a single or double bond.

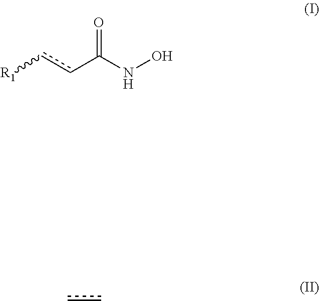

### CROSS-REFERENCE TO RELATED APPLICATIONS

This application claims the benefit of U.S. Provisional Application No. 63/453,541 filed Mar. 21, 2023, incorporated by reference herein.

### FIELD

The present disclosure relates to methods of increasing exogenous nucleic acid expression in microorganisms.

### BACKGROUND

Cells can modulate their level of chromatin condensation through histone and DNA methylation and histone deacetylation leading to the regulation of gene expression (Casas-Mollano et al., 2007). These post-translational modifications drive a repressive-condensed chromatin state that prevents transcription factor access to promoters (Shaver et al., 2010). Gene silencing by epigenetic modulations may have evolved in microorganisms as a defense mechanism to prevent expression of foreign DNA (Shaver et al., 2010; Neupert et al., 2020). Importantly, this mechanism of gene silencing impedes the expression of exogenous nucleic acids (e.g. transgenes) inserted into the genomes of genetically engineered microorganisms.

To counter gene silencing, specific hybrid promoters have been designed (Schroda et al., 2000; Specht et al., 2015), introns have been introduced into constructs (Baier et al., 2018) and transgene codons have been optimized. In addition, mutant strains with enhanced protein expression have been created following UV mutagenesis (Neupert et al., 2009; Kurniasih et al., 2016). These strains harbor mutations in histone deacetylase (HDAC) and DNA methyltransferase genes. These approaches may be of limited efficacy, as gene silencing may still occur (Neupert et al., 2009). Previous attempts to stimulate transgene expression in the model microorganism Chlamydomonas reinhardtii using histone deacetylase inhibitors have found either no effect on boosting expression, or a limited effect when the transgene is an antibiotic resistance gene required for cell survival and thus under strong selective pressure (Neupert et al., 2020; Kaginkar et al., 2021).

### SUMMARY

The present invention provides methods for improving the expression of an exogenous nucleic acid molecule in a microorganism.

In an embodiment of the present invention, there is provided a method of increasing expression of an exogenous nucleic acid in a microorganism comprising adding a hydroxamate-based histone deacetylase inhibitor to a culture medium of the microorganism.

### BRIEF DESCRIPTION OF THE DRAWINGS

The accompanying figures illustrate embodiments of the invention by way of example.

FIG. 1. Construct for transformation comprising the mVenus transgene.

FIG. 2. Selection of mVenus⁺ transformants by flow cytometry. Heatmap representation of the % mVenus⁺ cells (A) or mVenus mean fluorescent intensity (B) in each transformant, as assessed by flow cytometry. mVenus was acquired on the FL1 channel (filter at 525/10 nm) on 384 transformed antibiotic resistant microalgae transferred to liquid TAP medium in four 96 well plates for 6 days. Negative controls (wild-type; WT) are wells 95 and 96 of each plate. Crossed wells are transformant that did not grow in liquid medium. (C) Gating strategy and representative dot plots of a negative (left) and a positive (right) sample. (D) Correlation between mean fluorescence intensity and % of mVenus⁺ cells in 23 mVenus⁺ transformants and a wild-type strain re-assessed for mVenus production 6 weeks following the first screening.

FIG. 3. mVenus expression following treatment with silencing inhibitors. (A) Heatmap representations of the % of mVenus expressing cells (top) and of the mVenus delta mean fluorescence intensity (bottom) in transformed clones 16, 18, and 20 and wild-type (WT). Liquid culture in 96 wells were treated with DMSO (vector, 0.1%), Sirtinol (SIRT, 50 μM), vorinostat (SAHA, 2.5 μM), OSS_128167 (OSS, 100 μM), nicotinamide (NAD, 1 mM), sodium butyrate (ButNa, 5 mM), or a mix of all (Mix) for 24 hours. (B) Representative pseudocolor dot plots of mVenus levels in transformant or WT treated with histone deacetylase inhibitors or DMSO. *: p>0.05; **p>0.01, using one-way ANOVA Friedman's test with repeated measures.

FIG. 4. Hydroxamate-based histone deacetylase (HDAC) inhibitors are potent inducers of transgenic expression. Bar graph showing the mean % of mVenus⁺ cells+/−SD (A) or mVenus mean fluorescent intensity (B) acquired by flow cytometry at day 6 of culture in transformed clones 16, 17, 18, and 20 and wild-type (WT), treated with DMSO or inhibitors in 96 wells liquid culture. (C) mVenus mean fluorescence intensity curve plot over 5 days following inhibitors treatment in clones 16, 17, 18, and 20 and WT as measured using a microplate reader. mVenus fluorescence was acquired in scan mode and normalized to the OD₇₅₀ also acquired in scan mode. Finally, the levels of mVenus fluorescence in the WT control (calculated similarly) was subtracted from each sample.

FIG. 5. SAHA steadily increases transgene expression over culture period. Flask liquid cultures of transformed clones 16, 17, 18, and 20 and wild-type (WT) were treated with 5 μM of SAHA and grown for 12 days. The mean fluorescence intensity (MFI) of mVenus was assessed every day. (A) The MFI was measured on the overall culture using a microplate reader. (B) The MFI was measured using a flow cytometer. (C) The % of mVenus⁺ cells was measured across growth period using a flow cytometer. (D) Growth curve of cells measured by OD at 750 nm.

FIG. 6. SAHA induces a palmelloid phenotype in C. reinhardtii. (A) SAHA's effect on cell viability (membrane integrity) was verified using propidium iodide (PI) staining and analyzed on a flow cytometer (FL3 channel). (B) Representative microscopy picture of liquid culture in 96 wells of transformants treated with DMSO for 6 days. (C) Representative microscopy picture of liquid culture in 96 wells of transformants treated with 5 μM SAHA for 6 days. (D) Representative microscopy picture of liquid culture in 96 wells of transformants treated with 10 μM SAHA for 6 days. (E) Bar graph with means+/−SD of the % of palmelloid cells in transformants or wild-type (WT) cells following 5 μM SAHA treatment at day 3 and 6, as measured on a flow cytometer. FSC: forward scatter, SSC Side scatter. (F) Representative microscopy of liquid culture in 96 wells of transformants treated with 20, 40, and 80 μM SAHA for 6 days.

FIG. 7. Effect of SAHA on mRNA levels. mRNA levels were verified by RT-qPCR. Flask liquid cultures of transformed clones 16, 17, 18, and 20 and wild-type (WT), treated with 5 μM of SAHA and grown for 12 days were all tested at day 6. RT-qPCR bar graph result of mVenus mRNA relative expression (normalized on histone H3) in transformed clones 16, 17, 18, and 20 and WT treated with SAHA or DMSO (negative control).

FIG. 8. mVenus expression after 1 year of storage prior to treatment with histone deacetylase inhibitors. Heatmap representations of the % of mVenus expressing cells in transformed clones 16, 17, 18, and 20 and wild-type (WT). Liquid culture in 96 wells were treated with DMSO, sodium butyrate (SBut, 5 mM), cinnamic acid (CAci, 1 mM), lycorine (Lyco, 2.5 μM), OSS_128167 (OSS, 100 μM), sirtinol (Sirt, 50 μM), Vorinostat (SAHA, 5 M), or azacitidine (AZA, 1 μM) for 72 hours.

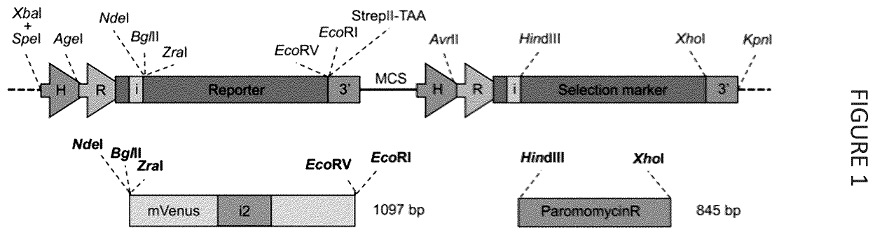

### DETAILED DESCRIPTION

The present disclosures relate to the unexpected discovery that hydroxamate-based histone deacetylase inhibitors provide a superior effect in increasing the expression of an exogenous nucleic acid in a microorganism. In the present examples, the exemplary microorganism Chlamydomonas reinhardtii, which is known to inhibit expression of transgenes by gene silencing, was transformed with an exemplary transgene encoding the mVenus fluorescent protein. It was surprisingly discovered that hydroxamate-based histone deacetylase inhibitors, such as the exemplary inhibitors SAHA (Vorinostat), Belinostat, Dacinostat, Panobinostat, substantially increase the expression of the transgene and with a greater effect than other histone deacetylase inhibitors.

Accordingly, the present disclosure provides a method of increasing expression of an exogenous nucleic acid in a microorganism comprising adding a hydroxamate-based histone deacetylase inhibitor to a culture medium of the microorganism.

Unless otherwise indicated, the definitions and embodiments described in this and other sections are intended to be applicable to all embodiments and aspects of the present disclosure herein described for which they are suitable as would be understood by a person skilled in the art.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

As used herein, the singular forms “a”, “an” and “the” include plural references unless the content clearly dictates otherwise.

### Exogenous Nucleic Acid

The present invention provides for the increased expression of an exogenous nucleic acid in a microorganism. A microorganism may be genetically engineered to comprise an exogenous nucleic acid by methods known in the art. As used herein, the term “genetically engineered microorganism” refers to a microorganism whose genetic material has been altered using molecular biology techniques such as but not limited to molecular cloning, recombinant DNA methods, transformation, DNA editing, and gene transfer. The genetically engineered microorganism includes a living modified microorganism, genetically modified microorganism or a transgenic microorganism. Genetic alteration includes addition, deletion, modification and/or mutation of genetic material.

The term “nucleic acid” or “nucleic acid molecule” or its derivatives, as used herein, refers to an unmodified DNA or RNA or modified DNA or RNA. The term “nucleic acid sequence” as used herein refers to a sequence of nucleoside or nucleotide monomers consisting of naturally occurring bases, sugars and intersugar (backbone) linkages and includes cDNA. The term also includes modified or substituted sequences comprising non-naturally occurring monomers or portions thereof. The nucleic acid sequences may be deoxyribonucleic acid sequences (DNA) or ribonucleic acid sequences (RNA) and may include naturally occurring bases including adenine, guanine, cytosine, thymidine and uracil. The sequences may also contain modified bases. Examples of such modified bases include aza and deaza adenine, guanine, cytosine, thymidine and uracil; and xanthine and hypoxanthine. The nucleic acid can be either double stranded or single stranded, and represents the sense or antisense strand. Further, the term “nucleic acid” includes the complementary nucleic acid sequences.

In some embodiments, the exogenous nucleic acid comprises a DNA sequence encoding a polypeptide or an RNA of interest and capable of expressing the polypeptide or RNA of interest in a microorganism. The DNA sequence encoding the polypeptide or RNA of interest in the exogenous nucleic acid may be operably linked to one or more regulatory elements known in the art to modulate expression of DNA sequences such as, for example, a promoter, a 5′ untranslated region (5′ UTR), a 3′ untranslated region (3′ UTR), an enhancer, an intron, and/or a terminator sequence. The term “operably-linked”, as used herein, refers to an arrangement of two or more components, wherein the components so described are in a relationship permitting them to function in a coordinated manner. For example, a transcriptional regulatory sequence or a promoter is operably-linked to a coding sequence if the transcriptional regulatory sequence or promoter facilitates aspects of the transcription of the coding sequence. The skilled person can readily recognize aspects of the transcription process, which include, but not limited to, initiation, elongation, attenuation and termination. In general, an operably-linked transcriptional regulatory sequence is joined in cis with the coding sequence, but it is not necessarily directly adjacent to it.

As used herein, the term “exogenous” refers to an element that has been introduced into a cell. An exogenous element can include a polypeptide or a nucleic acid. An exogenous nucleic acid is a nucleic acid that has been introduced into a cell, such as by a method of transformation. An exogenous nucleic acid may code for the expression of an RNA and/or a polypeptide. An exogenous nucleic acid may have been derived from the same species (homologous) or from a different species (heterologous). An exogenous nucleic acid may comprise a homologous sequence that is altered such that it is introduced into the cell in a form that is not normally found in the cell in nature. For example, an exogenous nucleic acid that is homologous may contain mutations, be operably linked to a different control region, or be integrated into a different region of the genome, relative to the endogenous version of the nucleic acid. An exogenous nucleic acid may be incorporated into the chromosomes of the transformed cell or into the plastid or mitochondrial DNA of the transformed cell, in one or more copies.

An exogenous nucleic acid molecule can be introduced into a cell by, for example, the stable integration of the nucleic acid molecule into the genome of a microorganism, wherein the genome includes nuclear DNA and plastid DNA, such as chloroplast DNA, or by the transient integration of the nucleic acid molecule into the microorganism. The introduction of a nucleic acid into a cell is also known in the art as transformation. The nucleic acid vectors may be introduced into the microorganism using techniques known in the art including, without limitation, agitation with glass beads, electroporation, agrobacterium-mediated transformation, an accelerated particle delivery method (i.e. particle bombardment), a cell fusion method or by any other method to deliver the nucleic acid molecule into a microorganism. In some embodiments, transformation comprises integration into the genome, including, without limitation, random integration by methods known in the art such as microprojectile bombardment or non-homologous end joining, or targeted integration by methods known in the art using DNA-editing enzymes such as CRISPR-Cas9. An exogenous nucleic acid may be incorporated into a vector for introduction into a microorganism. As used herein, the term “vector” or “nucleic acid vector” means a nucleic acid molecule, such as a plasmid, comprising regulatory elements and a site for introducing transgenic DNA (e.g. the exogenous nucleic acid), which is used to introduce said transgenic DNA into a microorganism. The transgenic DNA can encode a heterologous protein or RNA, which can be expressed in the microorganism. The transgenic DNA can be integrated into nuclear, mitochondrial or chloroplastic genomes through homologous or non-homologous recombination. The vector can contain a single, operably-linked set of regulatory elements that includes a promoter, a 5′ untranslated region (5′ UTR), an insertion site for transgenic DNA, a 3′ untranslated region (3′ UTR) and a terminator sequence. Vectors useful in the present methods are well known in the art.

The word “expression” as used herein refers to the translation of a polypeptide (for example a protein of interest) encoded by a nucleic acid (such as an exogenous nucleic acid) and/or transcription of an RNA transcript (for example, mRNA or an interfering RNA, such as microRNA (miRNA), short interfering RNA (siRNA), short hairpin RNA (shRNA) or Dicer-substrate small-interfering RNA (DsiRNA)) encoded by a nucleic acid (such as an exogenous nucleic acid). As used herein, “increasing expression” refers to increasing the amount of translated polypeptide and/or RNA transcript encoded by a nucleic acid (such as an exogenous nucleic acid) in a cell. The increased expression may be relative to a control microorganism that is not treated according to a method of the present invention. The increased expression may be relative to the microorganism prior to addition of a hydroxamate-based histone deacetylase inhibitor to a culture medium according to a method of the present invention.

As used herein, the phrase “protein of interest” or “polypeptide of interest” refers to any polypeptide that is desired to be expressed in the microorganism, including full length proteins, modified proteins, fragments of proteins and functional domains of proteins. In some embodiments, the protein of interest is an enzyme, antibody, DNA-binding protein, transcription regulating protein, membrane protein, nuclear receptor protein, nuclear localization signal protein, protein cofactor, intracellular structural protein, and fragments or derivatives thereof. In some embodiments, the protein of interest is a biosynthetic enzyme. “Biosynthetic enzyme” as used in herein refers to any protein catalyst involved in the process of making compounds of interest through chemical reactions. Examples of biosynthetic enzymes include enzymes involved in the biosynthesis of metabolites such as cannabinoids, alkaloids, glucosinolates, terpenes, steroids, saponins, flavonoids, tannins phenylpropanoids, lignins, coumarins, lignans, polyacetylenes, polyketides and tryptamine-derivatives. In some embodiments, the protein of interest is an enzyme of the cannabinoid biosynthetic pathway, such as those disclosed in WO/2019/210404 the disclosures of which are incorporated by reference herein. In some embodiments, the protein of interest is an enzyme of a tryptamine-derivative biosynthetic pathway, such as those disclosed in WO/2022/099424 the disclosures of which are incorporated by reference herein.

### Hydroxamate-Based Histone Deacetylase Inhibitors

Histone deacetylases (HDACs) are zinc hydrolases that modulate gene expression through deacetylation of the N-acetyl-lysine residues of histone proteins and other transcriptional regulators (Hassig & Schreiber, 1997). The present invention provides methods of increasing expression of an exogenous nucleic acid using hydroxamate-based histone deacetylase inhibitors.

As used herein, the term “hydroxamate-based histone deacetylase inhibitor” is used interchangeably with the term “hydroxamic acid-based histone deacetylase inhibitor”, as the person skilled in the art would understand that a hydroxamate is the conjugate base of a hydroxamic acid. As used herein, a “hydroxamate-based histone deacetylase inhibitor” is a compound comprising a chemical structure according to formula I that inhibits the histone deacetylase activity of a histone deacetylase.

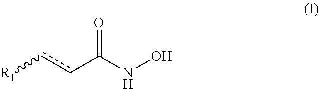

In some embodiments, R₁ is selected from the group consisting of

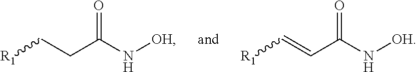

In some embodiments, R₁ is a cyclic or acyclic, substituted or unsubstituted, branched or unbranched aliphatic; a cyclic or acyclic, substituted or unsubstituted, branched or unbranched heteroaliphatic; a substituted or unsubstituted aryl; or a substituted or unsubstituted heteroaryl; and [symbol] represents a single or double bond.

In some embodiments, R₁ is an acyclic unsubstituted aliphatic. In some embodiments, R₁ is an acyclic substituted aliphatic. In some embodiments, Riis a cyclic unsubstituted aliphatic. In some embodiments, R₁ is a cyclic unsubstituted aliphatic. In some embodiments, R₁ is a branched substituted or unsubstituted aliphatic. In some embodiments, R₁ is an unbranched substituted or unsubstituted aliphatic.

In some embodiments, R₁ is a substituted or unsubstituted, branched or unbranched alkyl. In some embodiments, R₁ is a substituted or unsubstituted, branched or unbranched C₁₋₁₀ alkyl; a substituted or unsubstituted, branched or unbranched C₁₋₈ alkyl; a substituted or unsubstituted, branched or unbranched C₁₋₆ alkyl; or a substituted or unsubstituted, branched or unbranched C₁₋₄ alkyl. In some embodiments, R₁ is a methyl; ethyl; propyl; or butyl.

In some embodiments, Riis a substituted or unsubstituted alkenyl. In some embodiments, R₁ is a substituted or unsubstituted C₂₋₁₀ alkenyl; a substituted or unsubstituted C₂₋₈ alkenyl; a substituted or unsubstituted C₂₋₆ alkenyl; or a substituted or unsubstituted C₂₋₄ alkenyl.

In some embodiments, Riis a substituted or unsubstituted alkynyl. In some embodiments, R₁ is a substituted or unsubstituted C₂₋₁₀ alkynyl; a substituted or unsubstituted C₂₋₈ alkynyl; a substituted or unsubstituted C₂₋₆ alkynyl; a substituted or unsubstituted C₂₋₄ alkynyl.

In some embodiments, R₁ does not contain a stereocenter. In some embodiments, R₁ contains at least one stereocenter. In some embodiments, R₁ contains 1-5 stereocenters. In some embodiments, R₁ contains 1 stereocenter; 2 stereocenters; or 3 stereocenters. In some embodiments, the stereocenter has a (R)-configuration. In other embodiments, the stereocenter has a (S)-configuration.

In some embodiments, R₁ is substituted with halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; OR^{A}; —C(═O)R^{A}; —CO₂R^{A}; —C(═O)N(R^{A})₂; —CN; —SCN; —SR^{A}; —SOR^{A}; —SO₂R^{A}; —NO₂; —N(R^{A})₂; —NHC(O)R^{A}; or —C(R^{A})₃; wherein each occurrence of R^{A} is independently hydrogen; halogen; aliphatic; heteroaliphatic; acyl; aryl; heteroaryl; hydroxyl; alkoxy; aryloxy; alkylthioxy; arylthioxy; amino; alkylamino; dialkylamino; heteroaryloxy; or heteroarylthioxy. In some embodiments, R₁ is substituted with halogen. In some embodiments, R₁ is substituted with F, Cl, Br, or I.

In some embodiments, R₁ is substituted with —C(═O) R^{A}; wherein R^{A} is halogen; aliphatic; heteroaliphatic; acyl; aryl; heteroaryl; hydroxyl; alkoxy; aryloxy; alkylthioxy; arylthioxy; amino; alkylamino; dialkylamino; heteroaryloxy; or heteroarylthioxy. In some embodiments, R₁ is substituted with —C(═O) R^{A}; wherein R^{A} is substituted or unsubstituted aryl, arylalkyl, arylalkenyl, or arylalkynyl.

In some embodiments, R₁ is selected from the group consisting of:

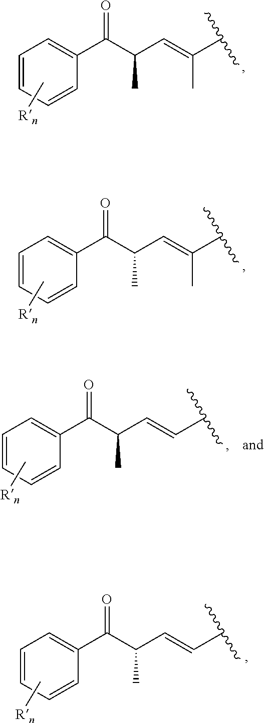

wherein n is an integer between 0-5, inclusive;
each occurrence of R′ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branded or unbranched acyl; substituted or unsubstituted, branched or unbranched aryl; substituted or unsubstituted, branched or unbranched heteroaryl; OR^{B}; —C(═O)R^{B}; —CO₂R^{B}; —C(═O)N(R^{B})₂; CN; —SCN; —SR^{B}; —SOR^{B}; —SO₂R^{B}; —NO₂; —N(R^{B})₂; —NHC(O)R^{B}; or —C(R^{B})₃; wherein each occurrence of R_{B} is independently hydrogen; halogen; aliphatic; heteroaliphatic; acyl; aryl; heteroaryl; hydroxyl; alkoxy; aryloxy; alkylthioxy; arylthioxy; amino; alkylamino; dialkylamino; heteroaryloxy; or heteroarylthioxy.

In some embodiments, R₁ is selected from the group consisting of:

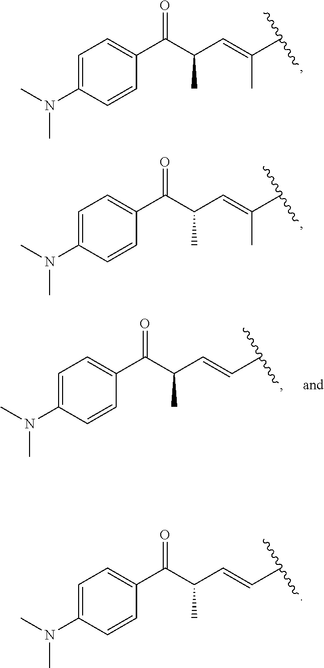 [SMILES: */C(C)=C/[C@@H](C)C(=O)c1ccc(N(C)C)cc1 ; */C(C)=C/[C@H](C)C(=O)c1ccc(N(C)C)cc1 ; */C=C/[C@@H](C)C(=O)c1ccc(N(C)C)cc1 ; */C=C/[C@H](C)C(=O)c1ccc(N(C)C)cc1]

In some embodiments, R₁ is substituted with —C(═O)R^{A}, wherein R^{A} is substituted or unsubstituted heteroaryl, heteroarylalkyl, heteroarylalkenyl, or heteroarylalkynyl.

In other embodiments, R₁ is selected from the group consisting of:

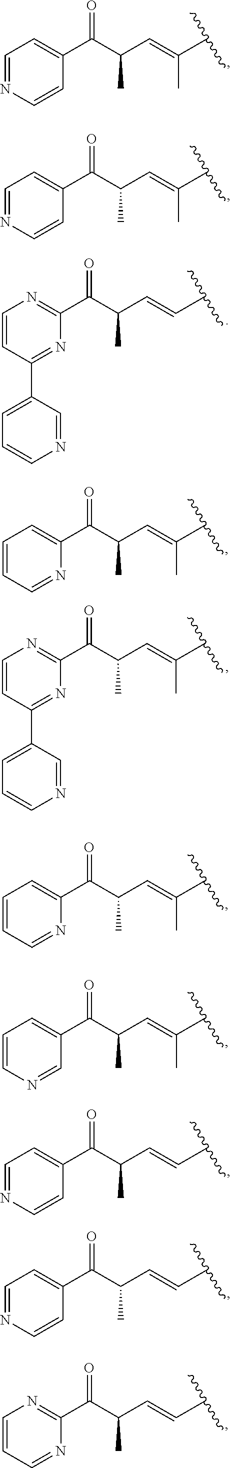

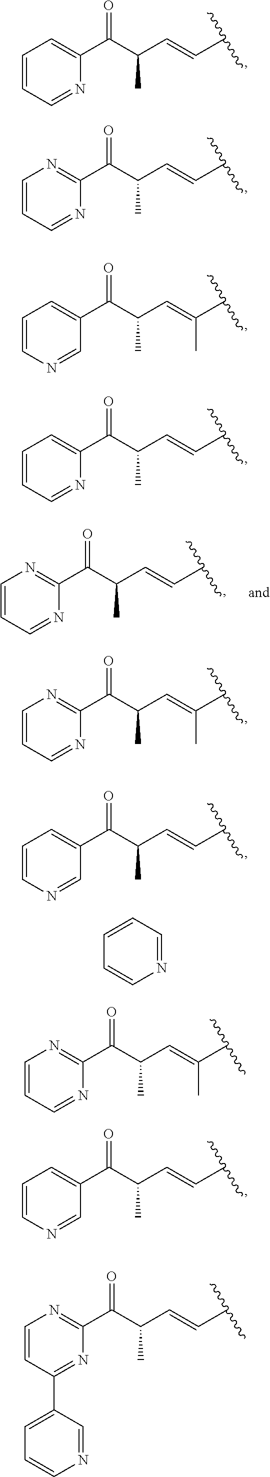

In some embodiments, R₁ is a substituted or unsubstituted aryl. In some, R₁ is an unsubstituted aryl. In some embodiments, R₁ is a substituted aryl. In some embodiments, R₁ is a 6-membered aryl; 8-membered aryl; or 10-membered aryl. In some embodiments, Riis an unsubstituted phenyl. In some embodiments, R₁ is a substituted phenyl. In some embodiments, R₁ is a monosubstituted phenyl; disubstituted phenyl; or trisubstituted phenyl. In some embodiments, R₁ is monocyclic ring system. In some embodiments, R₁ is a bicyclic ring system. In some embodiments, R₁ has one aromatic ring. In some embodiments, R₁ has two aromatic rings. In some embodiments, R₁ comprises phenyl. In some embodiments, R₁ comprises naphthyl. In some embodiments, R₁ comprises tetrahydronaphthyl.

In some embodiments, R₁ is

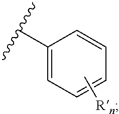

wherein n is an integer between 0-5, inclusive;
each occurrence of R′ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR^{B}; —C(═O)R^{B}; —CO₂R^{B}; —C(═O)N(R^{B})₂; —CN; —SCN; —SR^{B}; —SOR^{B}; —SO₂R^{B}; —NO₂; —N(R^{B})₂; —NHC(O)R^{B}; or —C(R^{B})₃; wherein each occurrence of R^{B} is independently hydrogen; halogen; aliphatic; heteroaliphatic; acyl; aryl; heteroaryl; hydroxyl; alkoxy; aryloxy; alkylthioxy; arylthioxy; amino; alkylamino; dialkylamino; heteroaryloxy; or heteroarylthioxy.

In some embodiments, the R′ groups are the same. In some embodiments, the R′ groups are different. In some embodiments, two R′ groups are taken together to form a ring. In some embodiments, two R′ groups are taken together to form a carbocyclic ring; a heterocyclic ring; an aromatic ring; an aryl ring; or a heteroaryl ring. In some embodiments, n is 1. In some embodiments, n is 2. In some embodiments, n is 3. In some embodiments, n is 4. In some embodiments, n is 5.

In an embodiment, R′ is

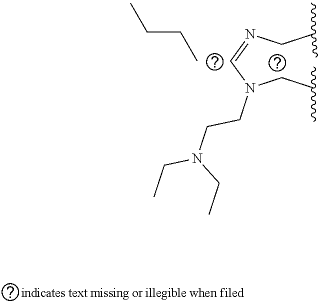

In some embodiments, R₁ is selected from the group consisting of:

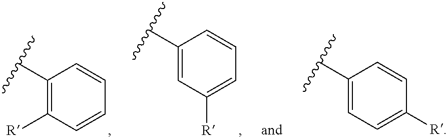

In some embodiments, R′ is selected from the group consisting of:

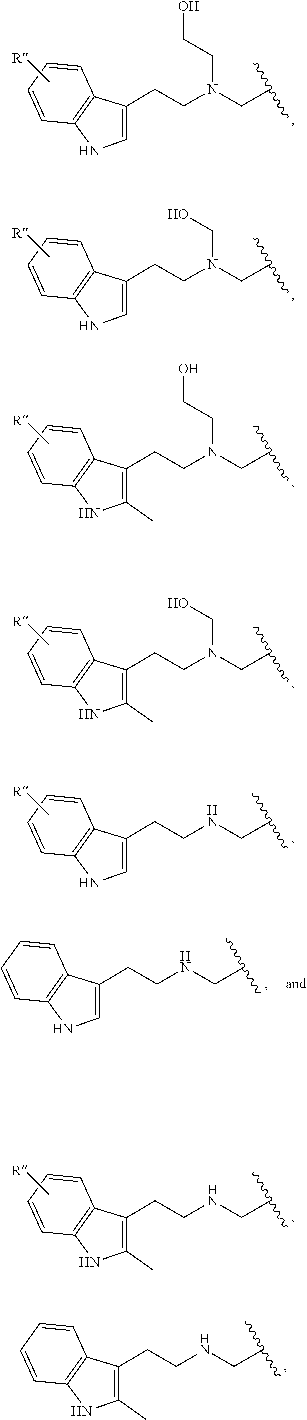

wherein each occurrence of R″ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR^{C}; —C(═O)R^{C}; —CO₂R^{C}; —C(═O)N(R^{C})₂; —CN; —SCN; —SR^{C}; —SOR^{C}; —SO₂R^{C}; —NO₂; —N(R^{C})₂; —NHC(O)R^{C}; or —C(R^{C})₃; wherein each occurrence of R^{B} is independently hydrogen; halogen; aliphatic; heteroaliphatic; acyl; aryl; heteroaryl; hydroxyl; alkoxy; aryloxy; alkylthioxy; arylthioxy; amino; alkylamino; dialkylamino; heteroaryloxy; or heteroarylthioxy.

In some embodiments, R′ is selected from the group consisting of:

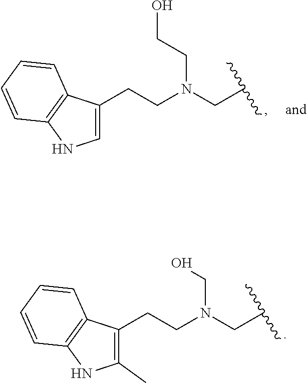 [SMILES: *CN(CCO)CCc1c[nH]c2ccccc12 ; *CN(CO)CCc1c(C)[nH]c2ccccc12]

In some embodiments, R′ is selected from the group consisting of:

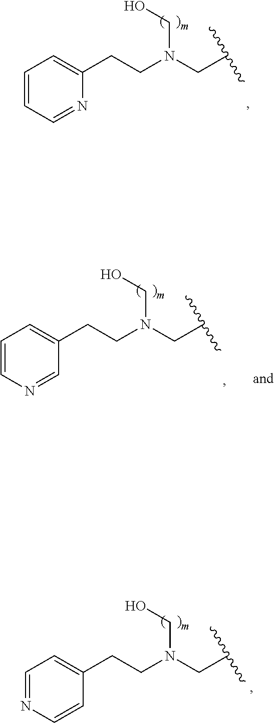

wherein m is an integer between 1 and 15, inclusive.

In some embodiments, R′ is selected from the group consisting of:

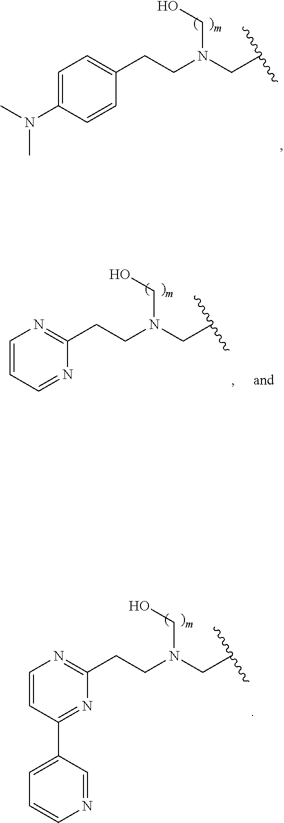

In some embodiments, R′ is selected from the group consisting of:

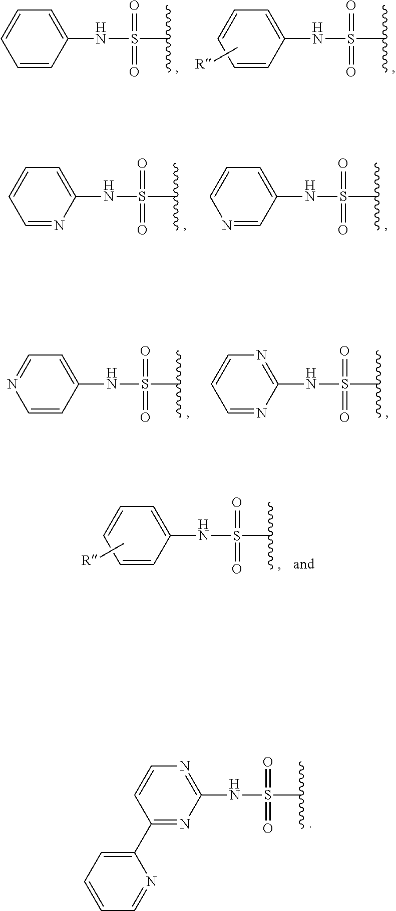

wherein R″ is as defined above.

In some embodiments, R′ is selected from the group consisting of:

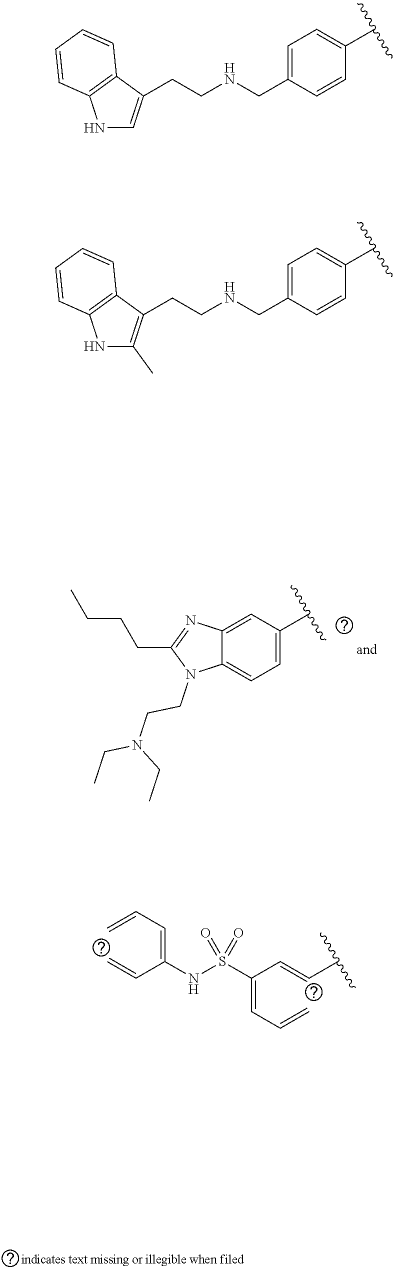 [SMILES: *c1ccc(CNCCc2c[nH]c3ccccc23)cc1 ; *c1ccc(CNCCc2c(C)[nH]c3ccccc23)cc1 ; *c1ccc2c(c1)nc(CCCC)n2CCN(CC)CC ; */C=C/C(=C\C=C)S(=O)(=O)N/C(C=C)=C/C=C]

In some embodiments, R₁ is selected from the group consisting of:

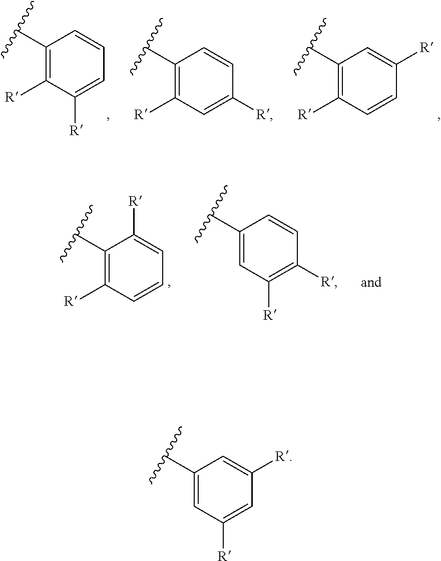

wherein R′ are as defined above.

In some embodiments, R₁ is selected from the group consisting of:

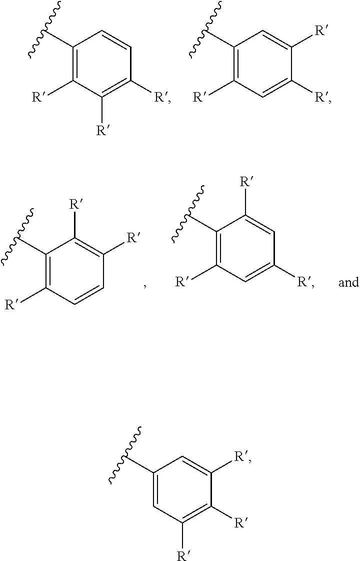

wherein R′ are as defined above.

In some embodiments, R₁ is a substituted or unsubstituted heteroaryl. In some embodiments, R₁ is an unsubstituted heteroaryl. In some embodiments, Riis a substituted heteroaryl. In some embodiments, R₁ is a nitrogen-containing heretoaryl. In some embodiments, R₁ is an O-containing heteroaryl. In some embodiments, R₁ is a S-containing heteroaryl. In some embodiments, R₁ is a 5-membered heteroaryl. In some embodiments, Riis a 6-membered heteroaryl. In some embodiments, R₁ is a bicyclic heteroaryl. In some embodiments, Riis a tricyclic heteroaryl.

In some embodiments, R₁ is selected from the group consisting of:

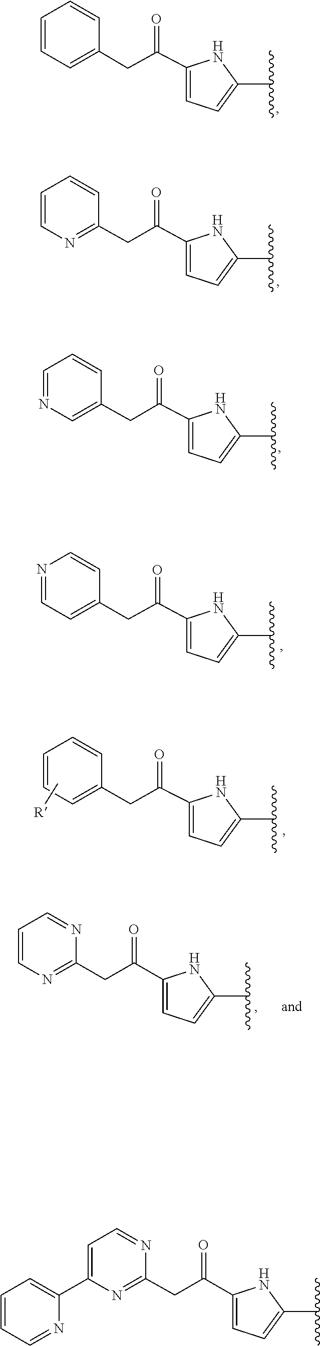

wherein R′ is as defined above.

In some embodiments, R₁ is

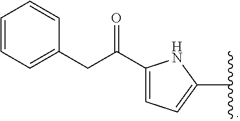 [SMILES: *c1ccc(C(=O)Cc2ccccc2)[nH]1]

In some embodiments, R₁ is-C(O) R′″, wherein R′″ is hydrogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR^{B}; —N(R^{B})₂; —NHC(O)R^{B}; or —C(R^{B})₃; wherein each occurrence of R_{B} is independently hydrogen; halogen; aliphatic; heteroaliphatic; acyl; aryl; heteroaryl; hydroxyl; alkoxy; aryloxy; alkylthioxy; arylthioxy; amino; alkylamino; dialkylamino; heteroaryloxy; or heteroarylthioxy.

In some embodiments, R₁ is selected from the group consisting of:

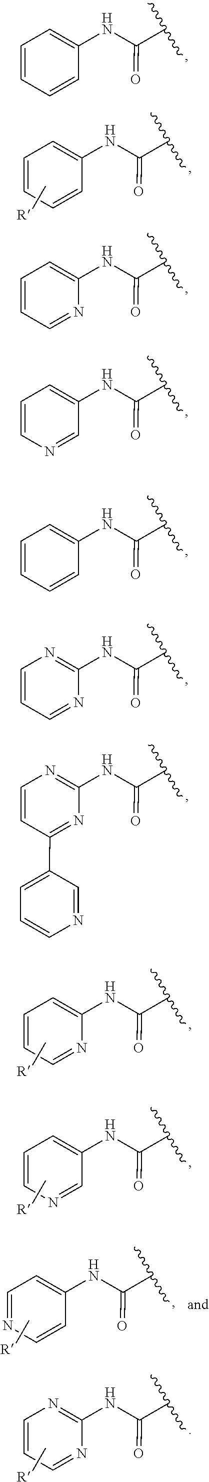

wherein R′ is as defined above.

In some embodiments, R₁ is selected from the group consisting of:

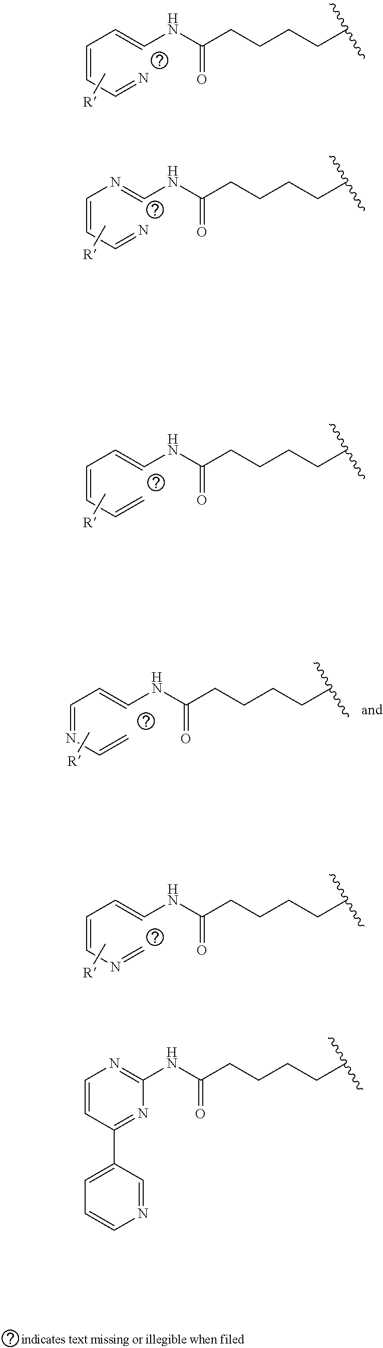

wherein R′ is as defined above.

In some embodiments, R₁ is selected from the group consisting of:

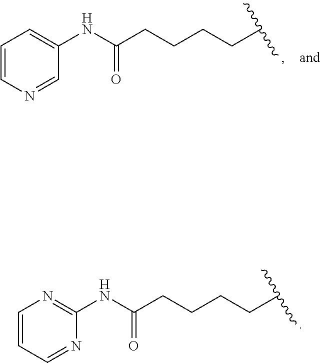 [SMILES: *CCCCC(=O)Nc1cccnc1 ; *CCCCC(=O)Nc1ncccn1]

In some embodiments, R₁ is selected from the group consisting of:

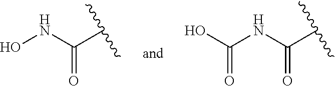 [SMILES: *C(=O)NO ; *C(=O)NC(=O)O]

In some embodiments, R₁ is:

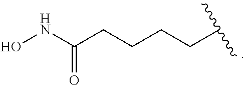 [SMILES: *CCCCC(=O)NO]

In some embodiments, R₁ is

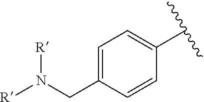

wherein each occurrence of R′ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR^{A}; —C(═O)R^{A}; —CO₂R^{A}; —C(═O)N(R^{A})₂; —CN; SCN; —SR^{A}; —SOR^{A}; —NO₂; —N(R^{A})₂; —NHC(O)R^{A}; or —C(R^{A})₃; wherein each occurrence of R^{A} is independently hydrogen; halogen; aliphatic, heteroaliphatic, acyl; aryl; heteroaryl; hydroxyl; alkoxy; aryloxy; alkylthioxy; arylthioxy; amino; alkylamino; dialkylamino; heteroaryloxy; heteroarylthioxy.

In some embodiments, R₁ is selected from a group consisting of:

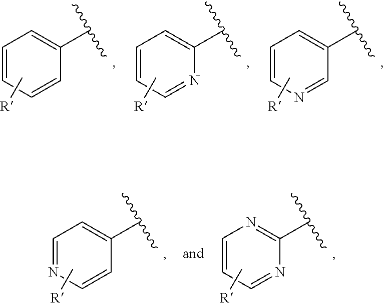

wherein each occurrence of R′ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR^{A}; —C(═O)R^{A}; —CO₂R^{A}; —C(═O)N(R^{A})₂; —CN; SCN; —SR^{A}; —SOR^{A}; —NO₂; —N(R^{A})₂; —NHC(O)R^{A}; or —C(R^{A})₃; wherein each occurrence of R^{A} is independently hydrogen; halogen; aliphatic, heteroaliphatic, acyl; aryl; heteroaryl; hydroxyl; alkoxy; aryloxy; alkylthioxy; arylthioxy; amino; alkylamino; dialkylamino; heteroaryloxy; heteroarylthioxy.

In some embodiments, R₁ is

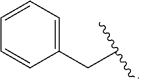 [SMILES: *Cc1ccccc1]

In some embodiments, R₁ is

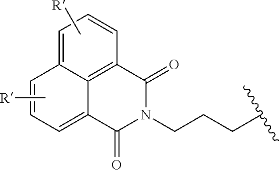

wherein R′ is as described above.

In some embodiments, R₁ is

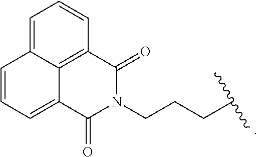 [SMILES: *CCCN1C(=O)c2cccc3cccc(c23)C1=O]

In some embodiments, R₁ is

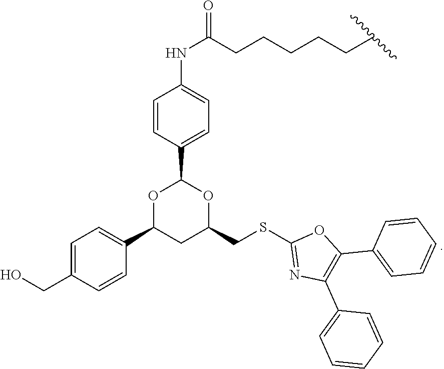 [SMILES: *CCCCCC(=O)Nc1ccc([C@H]2O[C@@H](CSc3nc(-c4ccccc4)c(-c4ccccc4)o3)C[C@@H](c3ccc(CO)cc3)O2)cc1]

In some embodiments, the hydroxamate-based histone deacetylase inhibitor is of the general formula A-B-C, wherein:
A is selected from the group consisting of cycloalkyls, unsubstituted and substituted aryls, heterocyclyls, amino aryls, and cyclopeptides;
B is selected from the group consisting of substituted C₄-C₈ alkylidenes, C₄-C₈ alkenylidenes, C₄-C₈ alkynylidenes, and -D-E-F)-, in which D and F are, independently, absent or represent a C₂-C₇alkylidene, a C₂-C₇alkenylidene or a C₂-C₇ alkynylidene; and E represents O, S, or NR′; in which R′ represents H, lower alkyl, lower alkenyl, lower alkynyl, aralkyl, aryl, or heterocyclyl; and
C is selected from the group consisting of:

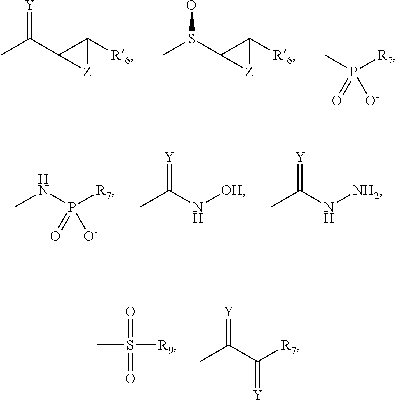

and boronic acid; wherein
Z represents O, S, or NR;
Y represents O or S;
R₅ represents hydrogen, alkyl, alkoxycarbonyl, aryloxycarbonyl, alkylsulfonyl, arylsulfonyl, or aryl;
R′₆ represents hydrogen, an alkyl, an alkenyl, an alkynyl, or an aryl;
R₇ represents a hydrogen, an alkyl, an aryl, an alkoxy, an aryloxy, an amino, a hydroxylamino, an alkoxylamino, or a halogen; and
R₉ represents a hydrogen, an alkyl, an aryl, a hydroxyl, an alkoxy, an aryloxy, or an amino.

In some embodiments, the hydroxamate-based deacetylase inhibitor is N-hydroxy-N′-phenyloctanediamide (also known as vorinostat, suberoylanilide hydroxamic acid, or SAHA), (2E)-N-Hydroxy-3-[3-(phenylsulfamoyl)phenyl]prop-2-enamide (also known as belinostat), (E)-N-hydroxy-3-[4-[[2-hydroxyethyl-[2-(1H-indol-3-yl)ethyl]amino]methyl]phenyl]prop-2-enamide (also known as dacinostat), (2E)-N-hydroxy-3-[4-({[2-(2-methyl-1H-indol-3-yl)ethyl]amino}methyl)phenyl]acrylamide (also known as panobinostat), (2E,4E,6R)-7-[4-(Dimethylamino)phenyl]-N-hydroxy-4,6-dimethyl-7-oxo-2,4-heptadienamide (also known as trichostatin A), N,N-dihydroxyoctanediamide (also known as suberohydroxamic acid, or SBHA) and/or (2E)-3-{2-Butyl-1-[2-(diethylamino)ethyl]-1H-1,3-benzimidazol-5-yl}-N-hydroxyprop-2-enamide (also known as pracinostat).

Hydroxamate-based histone deacetylase inhibitors may exist in particular geometric or stereoisomeric forms, including cis- and trans-isomers, R- and S-enantiomers, diastereomers, (D)-isomers, (L)-isomers, the racemic mixtures thereof, and other mixtures thereof. Additional asymmetric carbon atoms may be present in a substituent such as an alkyl group. All such isomers, as well as mixtures thereof, are intended to be included in the present disclosure.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios may be used. Those of ordinary skill in the art will readily appreciate that analogous ratios are contemplated for more complex isomer mixtures.

As used herein, the term “substituted” whether preceded by the term “optionally” or not, and substituents contained in formulas of this disclosure, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. The permissible substituents include acyclic and cyclic, branched and unbranched, carbocyclic and heterocyclic, aromatic and nonaromatic substituents of organic compounds. Heteroatoms such as nitrogen may have hydrogen substituents and/or any permissible substituents of organic compounds described herein which satisfy the valencies of the heteroatoms. As used herein, the word “stable” refers to compounds which can be maintained for a sufficient period of time to be useful for the purposes described herein.

As used herein, the word “acyl”, refers to a carbonyl-containing functionality, e.g., —C(═O)R, wherein R is an aliphatic, alicyclic, heteroaliphatic, heterocyclic, aryl, heteroaryl, (aliphatic) aryl, (heteroaliphatic) aryl, heteroaliphatic (aryl), or heteroaliphatic (heteroaryl) moiety, whereby each of the aliphatic, heteroaliphatic, aryl, or heteroaryl moieties is substituted or unsubstituted, or is a substituted (e.g., hydrogen or aliphatic, heteroaliphatic, aryl, or heteroaryl moieties) oxygen or nitrogen containing functionality (e.g., forming a carboxylic acid, ester, or amide functionality).

As used herein, the word “aliphatic”, includes both saturated and unsaturated, straight chain, namely, unbranched, or branched aliphatic hydrocarbons, which are optionally substituted with one or more functional groups. “Aliphatic” is intended herein to include, without limitation, alkyl, alkenyl, and alkynyl moieties. Thus, as used herein, the term “alkyl” includes straight and branched alkyl groups. An analogous convention applies to other generic terms such as “alkenyl”, “alkynyl”, and the like.

Furthermore, as used herein, the terms “alkyl”, “alkenyl”, “alkynyl”, and the like encompass both substituted and unsubstituted groups. In certain embodiments, as used herein, “lower alkyl” is used to indicate those alkyl groups (substituted, unsubstituted, branched or unbranched) having 1-6 carbon atoms. In certain embodiments, the alkyl, alkenyl, and alkynyl groups contain 1-20 aliphatic carbon atoms, 1-10 aliphatic carbon atoms, 1-8 aliphatic carbon atoms, 1-6 aliphatic carbon atoms, 1-4 aliphatic carbon atoms or 1-2 aliphatic carbon atoms. In certain other embodiments, the alkyl, alkenyl, and alkynyl groups employed in the invention contain 14 carbon atoms. Illustrative aliphatic groups include, without limitation, methyl, ethyl, n-propyl, isopropyl, allyl, n-butyl, sec-butyl, isobutyl, tert-butyl, n-pentyl, sec-pentyl, isopentyl, tert-pentyl, n-hexyl, sec-hexyl, moieties, which may bear one or more substituents. Alkenyl groups include, without limitation ethenyl, propenyl, butenyl, 1-methyl-2-buten-1-yl, and the like. Representative alkynyl groups include, without limitation, ethynyl, 2-propynyl (propargyl), 1-propynyl, and the like.

As used herein, the word “alicyclic” or “carbocyclic”, refers to compounds which combine the properties of aliphatic and cyclic compounds and include, without limitation, cyclic, or polycyclic aliphatic hydrocarbons and bridged cycloalkyl compounds, which are optionally substituted with one or more functional groups. “Alicyclic” is intended herein to include, without limitation, cycloalkyl, cycloalkenyl, and cycloalkynyl moieties, which are optionally substituted with one or more functional groups. Example alicyclic groups include, without limitation, cyclopropyl, —CH₂-cyclopropyl, cyclobutyl, —CH₂-cyclopentyl, cyclopentyl, —CH₂-cyclopentyl, cyclohexyl, —CH₂-cyclohexyl, cyclohexenylethyl, cyclohexanylethyl, norbornyl moieties, and the like, which may bear one or more substituents.

As used herein, the term “alkoxy” or “alkyloxyl” or “thioalkyl” means an alkyl group, as previously defined, attached to the parent molecular moiety through an oxygen atom or a sulfur atom. Examples of alkoxy, include, without limitation, methoxy, ethoxy, propoxy, isopropoxy, n-butoxy, tert-butoxy, neopentoxy, n-hexoxy and the like. Examples of thioalkyl include, without limitation, methylthio, ethylthio, propylthio, isopropylthio, n-butylthio, and the like.

The term “alkylamino” refers to a group having the structure —NHR′ wherein R′ is alkyl, as defined herein. The term “aminoalkyl” refers to a group having the structure NH2R′—, wherein R′ is alkyl, as defined herein. In certain embodiments, the alkyl group contains 1-20 aliphatic carbon atoms, 1-10 aliphatic carbon atoms, 1-8 aliphatic carbon atoms, 1-6 aliphatic carbon atoms, 1-4 aliphatic carbon atoms or 1-2 aliphatic carbon atoms. Examples of alkylamino include, without limitation, methylamino, ethylamino, iso-propylamino, n-propylamino, and the like.

Some examples of substituents of the above-described aliphatic (and other) moieties of compounds include, without limitation, aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br, I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ; wherein each occurrence of Rₓ independently includes, but is not limited to, aliphatic, alicyclic, heteroaliphatic, heterocyclic, aryl, heteroaryl, alkylaryl, or alkylheteroaryl, wherein any of the aliphatic, heteroaliphatic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted.

As used herein, the word “alkylidene”, refers to a substituted or unsubstituted, linear or branched saturated divalent radical consisting solely of carbon and hydrogen atoms, having from one to n carbon atoms, having a free valence “-” at both ends of the radical. In certain embodiments, the alkylidene moiety has 1 to 6 carbon atoms. As used herein, the word “alkenylidene” refers to a substituted or unsubstituted, linear or branched unsaturated divalent radical, consisting solely of carbon and hydrogen atoms, having from two to n carbon atoms, having a free valence “-” at both ends of the radical, and wherein the unsaturation is present only as double bonds and wherein a double bond can exist between the first carbon of the chain and the rest of the molecule. In certain embodiments, the alkenylidene moiety has 2 to 6 carbon atoms.

As used herein, the word “alkynylidene”, refers to a substituted or unsubstituted, linear or branched unsaturated divalent radical consisting solely of carbon and hydrogen atoms, having from two to n carbon atoms, having a free valence “-” at both ends of the radical, and wherein the unsaturation is present only as triple or double bonds and wherein a triple or double bond can exist between the first carbon of the chain and the rest of the molecule. In certain embodiments, the alkynylidene moiety has 2 to 6 carbon atoms.

Unless otherwise indicated, as used herein, the terms “alkyl”, “alkenyl”, “alkynyl”, “heteroalkyl”, “heteroalkenyl”, “heteroalkynyl”, “alkylidene”, “alkenylidene”, “alkynylidene”, -(alkyl) aryl, -(heteroalkyl) aryl, -(heteroalkyl) aryl, -(heteroalkyl) heteroaryl, and the like encompass substituted and unsubstituted, and linear and branched groups. Similarly, the terms “aliphatic”, “heteroaliphatic”, and the like encompass substituted and unsubstituted, saturated and unsaturated, and linear and branched groups. Similarly, the terms “cycloalkyl”, “heterocycle”, “heterocyclic”, and the like encompass substituted and unsubstituted, and saturated and unsaturated groups. Additionally, the terms “cycloalkenyl”, “cycloalkynyl”, “heterocycloalkenyl”, “heterocycloalkynyl”, “aromatic”, “heteroaromatic, “aryl”, “heteroaryl” and the like encompass both substituted and unsubstituted groups.

As used herein, the term “amino”, refers to a primary (—NH₂), secondary (—NHRₓ), tertiary (—NRₓR_{y}), or quaternary (—N⁺RₓR_{y}R_{z}) amine, where Rₓ, R_{y} and R_{z} are independently an aliphatic, alicyclic, heteroaliphatic, heterocyclic, aryl, or heteroaryl moiety, as defined herein. Examples of amino groups include, without limitation, methylamino, dimethylamino, ethylamino, diethylamino, diethylaminocarbonyl, methylethylamino, iso-propylamino, piperidino, trimethylamino, and propylamino.

As used herein, the phrase “aromatic moiety” refers to a stable mono- or polycyclic, unsaturated moiety having preferably 3-14 carbon atoms, each of which may be substituted or unsubstituted. In certain embodiments, the term “aromatic moiety” refers to a planar ring having p-orbitals perpendicular to the plane of the ring at each ring atom and satisfying the Huckel rule where the number of pi electrons in the ring is (4n+2), wherein n is an integer. A mono- or polycyclic, unsaturated moiety that does not satisfy one or all of these criteria for aromaticity is defined herein as “non-aromatic,” and is encompassed by the term “alicyclic.”

As used herein, the phrase “heteroaromatic moiety” refers to a stable mono- or polycyclic, unsaturated moiety having preferably 3-14 carbon atoms, each of which may be substituted or unsubstituted; and comprising at least one heteroatom selected from O, S, and N within the ring, namely, in place of a ring carbon atom. In certain embodiments, the term “heteroaromatic moiety” refers to a planar ring comprising at least on heteroatom, having p-orbitals perpendicular to the plane of the ring at each ring atom, and satisfying the Huckel rule where the number of pi electrons in the ring is (4n+2), wherein n is an integer. The aromatic and heteroaromatic moieties, as defined herein may be attached via an alkyl or heteroalkyl moiety and thus also include-(alkyl) aromatic, -(heteroalkyl) aromatic, -(heteroalkyl) heteroaromatic, and -(heteroalkyl) heteroaromatic moieties. Thus, as used herein, the phrases “aromatic or heteroaromatic moieties” and “aromatic, heteroaromatic, -(alkyl) aromatic, -(heteroalkyl) aromatic, -(heteroalkyl) heteroaromatic, and -(heteroalkyl) heteroaromatic” are interchangeable. Substituents include, without limitation, any of the previously mentioned substituents, namely, the substituents recited for aliphatic moieties, or for other moieties as disclosed herein.

As used herein, the word “aryl” refers to an unsaturated cyclic moiety comprising at least one aromatic ring. “Aryl” may refer to a mono- or bicyclic carbocyclic ring system having one or two aromatic rings including, without limitation, phenyl, naphthyl, tetrahydronaphthyl, indanyl, indenyl, and the like.

As used herein, the word “heteroaryl” refers to a cyclic aromatic radical having from five to ten ring atoms of which one ring atom is selected from S, O, and N; zero, one, or two ring atoms are additional heteroatoms independently selected from S, O, and N; and the remaining ring atoms are carbon, the radical being joined to the rest of the molecule via any of the ring atoms, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like.

The aryl and heteroaryl groups can be unsubstituted or substituted, wherein substitution includes replacement of one or more of the hydrogen atoms thereon independently with any one or more of the following moieties including, but not limited to aliphatic; alicyclic; heteroaliphatic; heterocyclic; aromatic; heteroaromatic; aryl; heteroaryl; alkylaryl; heteroalkylaryl; alkylheteroaryl; heteroalkylheteroaryl; alkoxy; aryloxy; heteroalkoxy; heteroaryloxy; alkylthio; arylthio; heteroalkylthio; heteroarylthio; F; Cl; Br, I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; and —NRₓ(CO)Rₓ; wherein each occurrence of Rₓ independently includes, without limitation, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl, heteroaryl, -(alkyl) aryl or (alkyl) heteroaryl substituents described above and herein may be substituted or unsubstituted. Additionally, any two adjacent groups taken together may represent a 4, 5, 6, or 7-membered substituted or unsubstituted alicyclic or heterocyclic moiety.

As used herein, the term “cycloalkyl”, refers specifically to groups having three to seven or three to ten carbon atoms in the form of a closed ring. Cycloalkyls include, without limitation, cyclopropyl, cyclobutyl, cyclopentyl, cyclohexyl, cycloheptyl, and the like, which, as in the case of aliphatic, alicyclic, heteroaliphatic or heterocyclic moieties, may optionally be substituted with substituents including, without limitation, aliphatic; alicyclic; heteroaliphatic; heterocyclic; aromatic; heteroaromatic; aryl; heteroaryl; alkylaryl; heteroalkylaryl; alkylheteroaryl; heteroalkylheteroaryl; alkoxy; aryloxy; heteroalkoxy; heteroaryloxy; alkylthio; arylthio; heteroalkylthio; heteroarylthio; F; Cl; Br, I; —OH; —NO₂; —CN; —CF₃; —CH₂CF₃; —CHCl₂—CH₂OH; —CH₂CH₂OH; —CH₂NH₂; —CH₂SO₂CH₃; —C(O) Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ; wherein each occurrence of Rₓ independently includes, without limitation, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl or heteroaryl substituents described above and herein may be substituted or unsubstituted.

As used herein, the term “heteroaliphatic,”, refers to aliphatic moieties in which one or more carbon atoms in the main chain have been substituted with a heteroatom. Thus, a heteroaliphatic group refers to an aliphatic chain which contains one or more oxygen, sulfur, nitrogen, phosphorus or silicon atoms, for example, in place of carbon atoms. Heteroaliphatic moieties may be linear or branched, and saturated or unsaturated. Heteroaliphatic moieties may be substituted by independent replacement of one or more of the hydrogen atoms thereon with one or more moieties including, without limitation, aliphatic; alicyclic; heteroaliphatic; heterocyclic; aromatic; heteroaromatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br, I; —OH; —NO₂; —CN; —CF₃; —CH₂CF₃; —CHCl₂; —CH₂OH; —CH₂CH₂OH; —CH₂NH₂; —CH₂SO₂CH₃; —C(O) Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; and —NRₓ(CO)Rₓ; wherein each occurrence of Rₓ independently includes, without limitation, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl or heteroaryl substituents described herein may be substituted or unsubstituted.

As used herein, the term “heterocycloalkyl,” “heterocycle,” or “heterocyclic,” refers to compounds which combine the properties of heteroaliphatic and cyclic compounds and include, without limitation, saturated and unsaturated mono- or polycyclic cyclic ring systems having 5-16 atoms wherein at least one ring atom is a heteroatom selected from O, S, and N (wherein the nitrogen and sulfur heteroatoms may be optionally oxidized), wherein the ring systems are optionally substituted with one or more functional groups, as defined herein. In certain embodiments, the term “heterocycloalkyl”, “heterocycle” or “heterocyclic” refers to a non-aromatic 5-, 6-, or 7-membered ring or a polycyclic group wherein at least one ring atom is a heteroatom selected from O, S, and N (wherein the nitrogen and sulfur heteroatoms may be optionally be oxidized), including, without limitation, a bi- or tri-cyclic group, comprising fused six-membered rings having between one and three heteroatoms independently selected from oxygen, sulfur and nitrogen, wherein (i) each 5-membered ring has 0 to 2 double bonds, each 6-membered ring has 0 to 2 double bonds and each 7-membered ring has 0 to 3 double bonds, (ii) the nitrogen and sulfur heteroatoms may be optionally be oxidized, (iii) the nitrogen heteroatom may optionally be quaternized, and (iv) any of the above heterocyclic rings may be fused to an aryl or heteroaryl ring. Representative heterocycles include, without limitation, furanyl, thiofuranyl, pyranyl, pyrrolyl, thienyl, pyrrolidinyl, pyrazolinyl, pyrazolidinyl, imidazolinyl, imidazolidinyl, piperidinyl, piperazinyl, oxazolyl, oxazolidinyl, isooxazolyl, isoxazolidinyl, dioxazolyl, thiadiazolyl, oxadiazolyl, tetrazolyl, triazolyl, thiatriazolyl, oxatriazolyl, thiadiazolyl, oxadiazolyl, morpholinyl, thiazolyl, thiazolidinyl, isothiazolyl, isothiazolidinyl, dithiazolyl, dithiazolidinyl, tetrahydrofuryl, and benzofused derivatives thereof. In certain embodiments, a “substituted heterocycle, or heterocycloalkyl or heterocyclic” group is utilized and as used herein, refers to a heterocycle, or heterocycloalkyl or heterocyclic group, as defined above, substituted by the independent replacement of one, two or three of the hydrogen atoms thereon with for example, without limitation, aliphatic; alicyclic; heteroaliphatic; heterocyclic; aromatic; heteroaromatic; aryl; heteroaryl; alkylaryl; heteroalkylaryl; alkylheteroaryl; heteroalkylheteroaryl; alkoxy; aryloxy; heteroaryloxy; heteroaryloxy; alkylthio; arylthio; heteroalkylthio; heteroarylthio; F; Cl; Br, I; —OH; —NO₂; —CN; —CF₃; —CH₂CF₃; —CHCl₂; —CH₂OH; —CH₂CH₂OH; —CH₂NH₂; —CH₂SO₂CH₃; —C(O) Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ; wherein each occurrence of Rₓ independently includes, without limitation, aliphatic, alicyclic; heteroaliphatic, heterocyclic, aromatic, heteroaromatic, aryl, heteroaryl, alkylaryl, alkylheteroaryl, heteroalkylaryl, or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl, or heteroaryl substituents described herein may be substituted or unsubstituted.

Any of the alicyclic or heterocyclic moieties described herein may comprise an aryl or heteroaryl moiety fused thereto.

### Microorganisms

In some embodiments, the microorganism is a microalga, a stramenopile, protist, or a fungus.

As used herein, the term “microalga” or “microalgae” and its derivatives, include photosynthetic and non-photosynthetic microorganisms that are eukaryotes. In some embodiments, the microalga is a species from Acutodesmus, Ankistrodesmus, Asteromonas, Aurantiochytrium, Auxenochlorella, Basichlamys, Botryococcus, Botryokoryne, Borodinella, Brachiomonas, Catena, Carteria, Chaetophora, Characiochloris, Characiosiphon, Chlainomonas, Chlamydomonas, Chlorella, Chlorochytrium, Chlorococcum, Chlorogonium, Chloromonas, Closteriopsis, Dictyochloropsis, Dunaliella, Ellipsoidon, Eremosphaera, Eudorina, Floydiella, Friedmania, Haematococcus, Hafniomonas, Heterochlorella, Gonium, Halosarcinochlamys, Koliella, Lobocharacium, Lobochlamys, Lobomonas, Lobosphaera, Lobosphaeropsis, Marvania, Monoraphidium, Myrmecia, Nannochloris, Nannochloropsis, Oocystis, Oogamochlamys, Pabia, Pandorina, Parietochloris, Phacotus, Phaeodactylum, Platydorina, Platymonas, Pleodorina, Polulichloris, Polytoma, Polytomella, Prasiola, Prasiolopsis, Prasiococcus, Prototheca, Pseudochlorella, Pseudocarteria, Pseudotrebouxia, Pteromonas, Pyrobotrys, Rosenvingiella, Scenedesmus, Schizochytrium, Spirogyra, Stephanosphaera, Tetrabaena, Tetraedron, Tetraselmis, Thalassiosira, Thraustochytium, Trebouxia, Trochisciopsis, Ulkenia, Viridiella, Vitreochlamys, Volvox, Volvulina, Vulcanochloris, Watanabea, Yamagishiella, Euglena, Isochrysis, or Nannochloropsis. In some embodiments, the microalgae is a species of the class Chlorophyceae. In some embodiments, the microalgae is a species of the order Chlamydomonadales. In some embodiments, the microalgae is a species of the family Chlamydomonadaceae. In some embodiments, the microalgae is a species of the genus Chlamydomonas. In some embodiments, the microalga is Chlamydomonas reinhardtii, Chlorella vulgaris, Chlorella sorokiniana, Chlorella protothecoides, or Schizochytrium limacinum. In some embodiments, the microalga is a diatom, optionally Phaeodactylum tricornutum or Thalassiosira pseudonana.

In some embodiments, the stramenopile is a species from Coscinodiscophyceae, Bacillariphyceae, Eustigmatophyceae, or Labyrinthylomycetes. In some embodiments, the stramenopile is a species from Thalassiosirales, Baccilariales, Eustigmatales, or Labyrinthulales. In some embodiments, the stramenopile is a species from Thalassiosira, Phaeodactylum, Nannochloropsis, Schizochytrium Aurantiochytrium, Thraustochytrium, or Ulkenia. In some embodiments, the stramenopile is Phaeodactylum tricornutum or Thalassiosira pseudonana.

In some embodiments, the microorganism is a protist, for example from the genera Dictyostelium, Tetrahymena, Emiliania.

In some embodiments, the microorganism is a fungus, for example from the genera Aspergillus, Saccharomyces, Schizosaccharomyces, or Fusarium.

### Culture of Microorganisms

Microorganisms may be cultured in conditions that are permissive to their growth. It is known that photosynthetic microorganisms are capable of carbon fixation wherein carbon dioxide (which is not a fixed carbon source) is fixed into organic molecules such as sugars using energy from a light source. The fixation of carbon dioxide using energy from a light source is photosynthesis. Suitable sources of light for the provision of energy in photosynthesis include sunlight and artificial lights. Photosynthetic microorganisms are capable of growth and/or metabolism without a fixed carbon source. Photosynthetic microorganisms can fix carbon dioxide from a variety of sources, including atmospheric carbon dioxide, industrially-discharged carbon dioxide (e.g. flue gas and flaring gas), and from soluble carbonates (e.g. NaHCO3 and Na2CO3), (Singh & Singh, 2014). A non-fixed carbon source such as carbon dioxide can be added to a culture of photosynthetic microorganisms by injection or by bubbling of a carbon dioxide gas mixture into the culture medium. Photosynthetic growth is a form of autotrophic growth, wherein a microorganism is able to produce organic molecules on its own using an external energy source such as light. This is in contrast to heterotrophic growth, wherein a microorganism must consume organic molecules for growth and/or metabolism. Heterotrophic organisms therefore require a fixed carbon source for growth and/or metabolism. Some photosynthetic organisms are capable of mixotrophic growth, wherein the microorganism fixes carbon by photosynthesis while also consuming fixed carbon sources. In mixotrophic growth, the autotrophic metabolism is integrated with a heterotrophic metabolism that oxidizes reduced carbon sources available in the culture medium. Photosynthetic microorganisms are commonly cultivated in mixotrophic conditions by adding fixed carbon sources as described herein to the culture medium. Common sources of fixed carbon that are used include glucose, ethanol, or waste products from industry such as acetate or glycerol (Cecchin et al. 2018). Microorganisms such as microalgae may be cultured using methods and conditions known in the art (see, e.g., Biofuels from Algae, eds. Pandey et al., 2014, Elsevier, ISBN 978-0-444-59558-4). Some microorganisms are capable of chemoautotrophic growth, Similar to photosynthetic microorganisms, chemoautotrophic organisms are capable of carbon dioxide fixation but using energy derived from chemical sources (e.g. hydrogen sulfide, ferrous iron, molecular hydrogen, ammonia) rather than light.

Microorganisms can be grown in organic conditions without the use of chemicals or additives that contravene the standards for organically-produced products. Microorganisms can be grown organically, for example, by growing them in conditions that comply with jurisdictional standards such as the standards set by the United States (US Organic Food Production Act; USDA National Organic Program Certification; USDA Organic Regulations), the European Union (Regulation No 834/2007 prior to Jan. 1, 2021; Regulation 2018/848 from Jan. 1, 2021), and Canada (Canadian Food Inspection Agency Canadian Organic Standards). Growing microorganisms in organic conditions permits the production of organic natural products in microorganisms.

Many media for the culture of microorganisms are known in the art and are suitable for use in the present invention including, but not limited to, F/2 media, L1 media, Tris acetate phosphate (TAP), or Bold's Basal Medium (BBM), as disclosed in de Carvalho et al., Biofuels from Algae (Second Edition), Elsevier, 2019, 33-50.

The culture of microorganisms comprises distinct phases. Initially, a new culture of microorganisms exists in a lag phase, wherein the microorganisms are adapting to the culture conditions. In the lag phase, the microorganisms are active but cell division is low or is not occurring. Subsequent to the lag phase, the rate of cell division accelerates as the culture enters the exponential phase, wherein nutrients are typically in excess and the microorganisms multiply exponentially at a constant rate. When a culture of microorganisms reaches a high density, the availability of nutrients begins to decline, and/or metabolic waste products accumulate in the culture environment, causing the culture to enter a stationary phase wherein the rate of cell division begins to decline. In the stationary phase the rate of cell division will slow down and the population of the culture reaches a plateau. If a culture is allowed to continue for long enough in the stationary phase then a death phase will begin wherein microorganisms die due to nutrient starvation and/or the toxicity of metabolic waste products, and the population of the culture declines. It is within the ability of the skilled person to determine the phase of a culture of microorganisms by methods known in the art, for example by using optical density to determine the cell density within the culture.

In some embodiments, the hydroxamate-based histone deacetylase inhibitor is added in the stationary phase of growth of the microorganism. In some embodiments, the microorganism is cultured with the hydroxamate-based histone deacetylase inhibitor for about 0.25 to about 10 days, about 0.5 to about 9 days, about 0.75 to about 8 days, about 1.25 to about 7 days, about 1.5 to about 6 days, about 1.75 to about 5 days, or about 2 to about 4 days. In some embodiments, the microorganism is cultured with the hydroxamate-based histone deacetylase inhibitor for about 1.5 days, about 1.75 days, about 2 days, about 2.25 days, about 2.5 days, about 2.75 days about 3 days, about 3.25 days, or about 3.5 days.

In some embodiments, the hydroxamate-based histone deacetylase inhibitor is added in the lag phase of growth of the microorganism. In some embodiments, the hydroxamate-based histone deacetylase inhibitor is added in the exponential phase of growth of the microorganism. In some embodiments, the microorganism is cultured with the hydroxamate-based histone deacetylase inhibitor for about 0.25 to about 14 days, about 0.5 to about 13 days, about 0.75 to about 12 days, about 1 to about 11 days, about 2 to about 10 days, about 3 to about 9 days, about 4 to about 8 days, or about 5 to about 7 days. In some embodiments, the microorganism is cultured with the hydroxamate-based histone deacetylase inhibitor for about 3.5 days, about 3.75 days, about 4 days, about 4.25 days, about 4.5 days, about 4.75 days, about 5 days, about 5.25 days, about 5.5 days, about 5.75 days, about 6 days, about 6.25 days, about 6.5 days, about 6.75 days, about 7 days, about 7.25 days, about 7.5 days, about 7.75 days, about 8 days, about 8.25 days or about 8.5 days.

In some embodiments, the hydroxamate-based histone deacetylase inhibitor is added to the culture medium to arrive at a concentration of 0.001 to 100 μM, a concentration of 0.005 to 100 μM, a concentration of 0.01 to 100 μM, a concentration of 0.1 to 80 μM, a concentration of 1 to 40 μM, a concentration of 2 to 20 μM, a concentration of 2.5 to 10 μM, or a concentration of 5 to 7.5 μM in the culture medium. In some embodiments, the hydroxamate-based histone deacetylase inhibitor is added to the culture medium to arrive at a concentration of 0.5 μM, 0.75 μM, 1 μM, 1.25 μM, 1.5 M, 1.75 M, 2 μM, 2.25 M, 2.5 μM, 2.75 μM, 3 μM, 3.25 μM, 3.5 μM, 3.75 μM, 4 μM, 4.25 μM, 4.5 μM, 4.75 μM, 5 μM, 5.25 μM, 5.5 μM, 5.75 μM, 6 μM, 6.25 μM, 6.5 μM, 6.75 μM, 7 μM, 7.25 μM, 7.5 μM, 7.75 μM, 8 μM, 8.25 μM, 8.5 μM, 8.75 μM, 9 μM, 9.25 μM, 9.5 μM, 9.75 μM, 10 μM, 15 μM, 20 μM, 25 μM, 30 μM, 35 μM, 40 μM, 45 μM, 50 μM, 55 μM, 60 μM, 65 μM, 70 μM, 75 μM, 80 μM, 85 μM, 90 μM, 95 μM, or 100 μM in the culture medium.

In some embodiments, the methods of the present invention comprise comparing the cell growth kinetics and/or cell viability of the microorganism to a control. The word “control” as used herein may refer to a microorganism cultured under the same conditions but in the absence of a hydroxamate-based deacetylase inhibitor. In some embodiments, the cell growth kinetics and/or cell viability of the microorganism cultured according to methods of the present invention is at least 60% of a control, at least 65% of a control, at least 70% of a control, at least 75% of a control, at least 80% of a control, at least 81% of a control, at least 82% of a control, at least 83% of a control, at least 84% of a control, at least 85% of a control, at least 86% of a control, at least 87% of a control, at least 88% of a control, at least 89% of a control, at least 90% of a control, at least 95% of a control, or at least 99% of a control.

In some embodiments, the methods of the present invention comprise determining cell growth kinetics by measuring optical density. Optical density can be measured by methods known in the art, including, without limitation, measurement of the absorbance at either 600 or 750 nm.

In some embodiments, the methods of the present invention comprise determining cell viability by membrane integrity confirmation using propidium iodide staining. Propidium iodide staining and cell viability determinations may be conducted using techniques known in the art including, without limitation, flow cytometry.

In some embodiments, the microorganism was stored after transformation with the exogenous nucleic acid and prior to use in methods of the present invention. As used herein, the word “stored” and its derivatives, refers to keeping the microorganism under conditions that slow or arrest growth, such that the microorganism remains in a viable state and may be restored to active culture. As used herein, the term “stored on or in a culture or a storage medium” includes storage in solid, semi-solid or liquid media known in the art, including without limitation, agar or broth. The media may also contain cryoprotectants know in the art, such as glycerol or DMSO. As used herein, the word “cryopreservation” means the process wherein microorganisms are frozen for storage. As used herein, the word “lyophilization” refers to a low temperature dehydration process wherein liquid solvent is removed from samples by sublimation.

In some embodiments, the microorganism has been stored for at least a day, at least three days, at least five days, at least a week, at least two weeks, at least three weeks, at least 1 month, at least 2 months, at least 3 months, at least 4 months, at least 5 months, at least 6 months, at least 7 months, at least 8 months, at least 9 months, at least 10 months, at least 11 months, at least a year, at least two years, at least five years, or at least ten years after transformation and prior to use in methods of the present invention.

Particular embodiments of the disclosure include, without limitation, the following:

1. A method of increasing expression of an exogenous nucleic acid in a microorganism comprising adding a hydroxamate-based histone deacetylase inhibitor to a culture medium of the microorganism.
2. The method of embodiment 1, wherein the hydroxamate-based deacetylase inhibitor has the structure of:

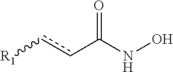

wherein
R₁ is a cyclic or acyclic, substituted or unsubstituted, branched or unbranched aliphatic; a cyclic or acyclic, substituted or unsubstituted, branched or unbranched heteroaliphatic; a substituted or unsubstituted aryl, or a substituted or unsubstituted heteroaryl; and [symbol]
   represents a single or double bond.
   3. The method of embodiment 2, wherein formula (I) is selected from the group consisting of:

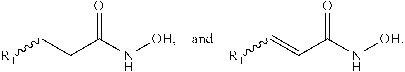

4. The method of embodiment 2, wherein R₁ is a cyclic or acyclic, substituted or unsubstituted, branched or unbranched aliphatic.
5. The method of embodiment 2, wherein R₁ is a cyclic, substituted or unsubstituted, branched or unbranched aliphatic.
6. The method of embodiment 2, wherein R₁ is an acyclic, substituted or unsubstituted, branched or unbranched aliphatic.
7. The method of embodiment 2, wherein R₁ is a substituted or unsubstituted C₁₋₁₀ alkyl.
8. The method of embodiment 2, wherein R₁ is a substituted or unsubstituted C₁₋₆ alkyl.
9. The method of embodiment 2, wherein R₁ is a substituted or unsubstituted C₁₋₄ alkyl.
10. The method of embodiment 2, wherein R₁ is a substituted or unsubstituted C₂₋₁₀ alkenyl.
11. The method of embodiment 2, wherein R₁ is a substituted or unsubstituted C₂₋₆ alkenyl.
12. The method of embodiment 2, wherein R₁ is a substituted or unsubstituted C₂₋₄ alkenyl.
13. The method of embodiment 2, wherein R₁ contains at least one stereocenter.
14. The method of embodiment 2, wherein R₁ is a cyclic or acyclic, substituted or unsubstituted, branched or unbranched heteroaliphatic.
15. The method of embodiment 2, wherein R₁ is a cyclic, substituted or unsubstituted, branched or unbranched heteroaliphatic.
16. The method of embodiment 2, wherein R₁ is an acyclic, substituted or unsubstituted, branched or unbranched heteroaliphatic.
17. The method of embodiment 2, wherein R₁ is selected from the group consisting of:

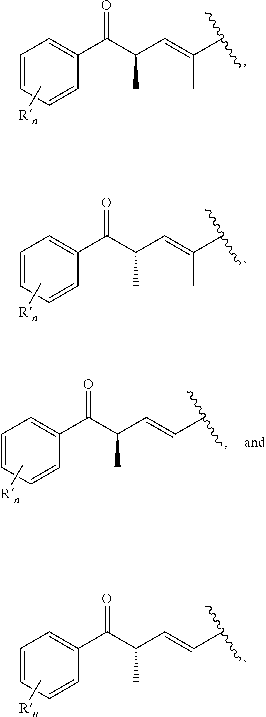

wherein
n is an integer between 0 and 5, inclusive; and
each occurrence of R′ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branded or unbranched acyl; substituted or unsubstituted, branched or unbranched aryl; substituted or unsubstituted, branched or unbranched heteroaryl; —OR^{B}; —C(═O)R^{B}; —CO₂R^{B}; —C(═O)N(R^{B})₂; —CN; —SCN; —SR^{B}; —SOR^{B}; —SO₂R^{B}; —NO₂; —N(R^{B})₂; —NHC(O)R^{B}; or —C(R^{B})₃; wherein each occurrence of R_{B} is independently hydrogen; halogen; aliphatic; heteroaliphatic; acyl; aryl; heteroaryl; hydroxyl; alkoxy; aryloxy; alkylthioxy; arylthioxy; amino; alkylamino; dialkylamino; heteroaryloxy; or heteroarylthioxy.
   18. The method of embodiment 2, wherein R₁ is selected from the group consisting of:

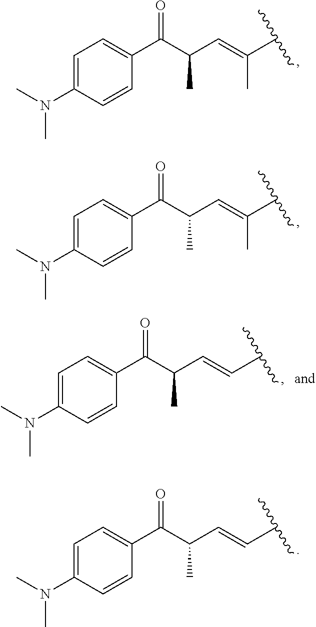 [SMILES: */C(C)=C/[C@@H](C)C(=O)c1ccc(N(C)C)cc1 ; */C(C)=C/[C@H](C)C(=O)c1ccc(N(C)C)cc1 ; */C=C/[C@@H](C)C(=O)c1ccc(N(C)C)cc1 ; */C=C/[C@H](C)C(=O)c1ccc(N(C)C)cc1]

19. The method of embodiment 2, wherein R₁ is selected from the group consisting of:

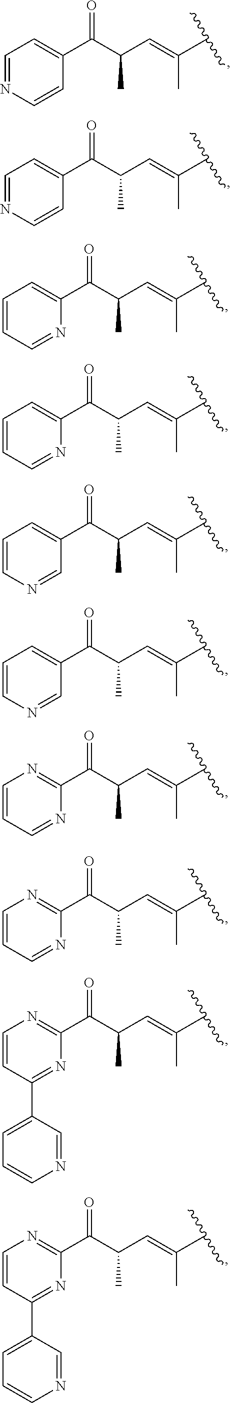

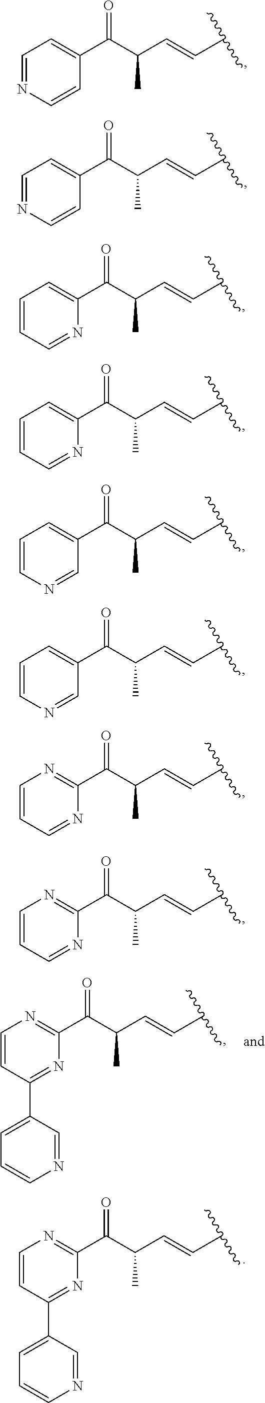

20. The method of embodiment 2, wherein R₁ is a substituted or unsubstituted aryl.
21. The method of embodiment 20, wherein R₁ is an unsubstituted aryl.
22. The method of embodiment 20, wherein R₁ is a substituted aryl.
23. The method of embodiment 20, wherein R₁ is a 6-membered aryl.
24. The method of embodiment 20, wherein R₁ is a 8-membered aryl.
25. The method of embodiment 20, wherein R₁ is a 10-membered aryl.
26. The method of embodiment 20, wherein R₁ is an unsubstituted phenyl.
27. The method of embodiment 20, wherein R₁ is a substituted phenyl.
28. The method of embodiment 20, wherein R₁ is:

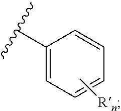

wherein
n is an integer between 0 and 5, inclusive; and
each occurrence of R′ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR^{B}; —C(═O)R^{B}; —CO₂R^{B}; —C(═O)N(R^{B})₂; —CN; —SCN; —SR^{B}; —SOR^{B}; —SO₂R^{B}; —NO₂; —N(R^{B})₂; —NHC(O)R^{B}; or —C(R^{B})₃; wherein each occurrence of R_{B} is independently hydrogen; halogen; aliphatic; heteroaliphatic; acyl; aryl; heteroaryl; hydroxyl; alkoxy; aryloxy; alkylthioxy; arylthioxy; amino; alkylamino; dialkylamino; heteroaryloxy; or heteroarylthioxy.
   29. The method of embodiment 28, wherein n is 1.
   30. The method of embodiment 28, wherein R′ is

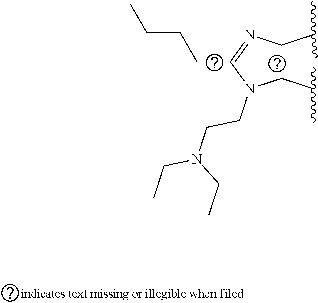

31. The method of embodiment 29, wherein R₁ is selected from the group consisting of:

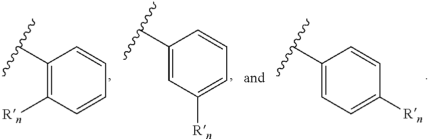

32. The method of embodiment 31, wherein R′ is selected from the group consisting of:

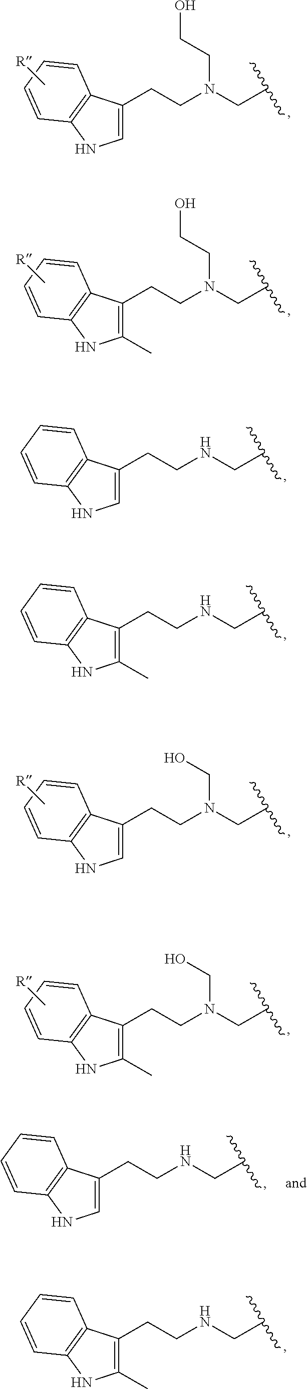

wherein each occurrence of R″ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR^{A}; —C(═O)R^{A}; —CO₂R^{A}; —C(═O)N(R^{A})₂; —CN; —SCN; —SR^{A}; —SOR^{A}; —SO₂R^{A}; —NO₂; —N(R^{A})₂; —NHC(O)R^{A}; or —C(R^{A})₃; wherein each occurrence of R^{A} is independently hydrogen; halogen; aliphatic; heteroaliphatic; acyl; aryl; heteroaryl; hydroxyl; alkoxy; aryloxy; alkylthioxy; arylthioxy; amino; alkylamino; dialkylamino; heteroaryloxy; or heteroarylthioxy.
33. The method of embodiment 31, wherein R′ is selected from the group consisting of:

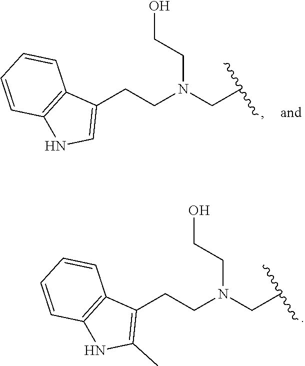 [SMILES: *CN(CCO)CCc1c[nH]c2ccccc12 ; *CN(CCO)CCc1c(C)[nH]c2ccccc12]

34. The method of embodiment 31, wherein R′ is selected from the group consisting of:

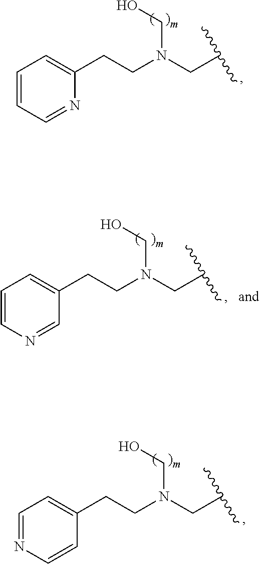

wherein m is an integer between 1 and 6, inclusive.
35. The method of embodiment 31, wherein R′ is selected from the group consisting of:

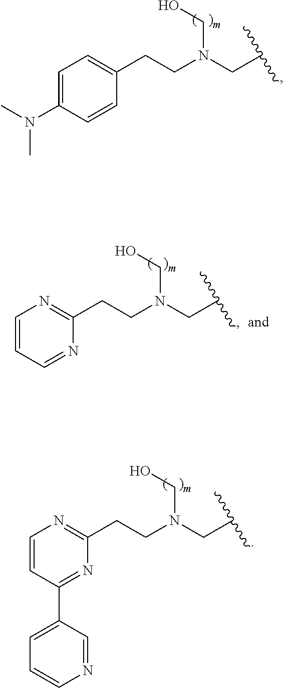

36. The method of embodiment 31, wherein R′ is selected from the group consisting of:

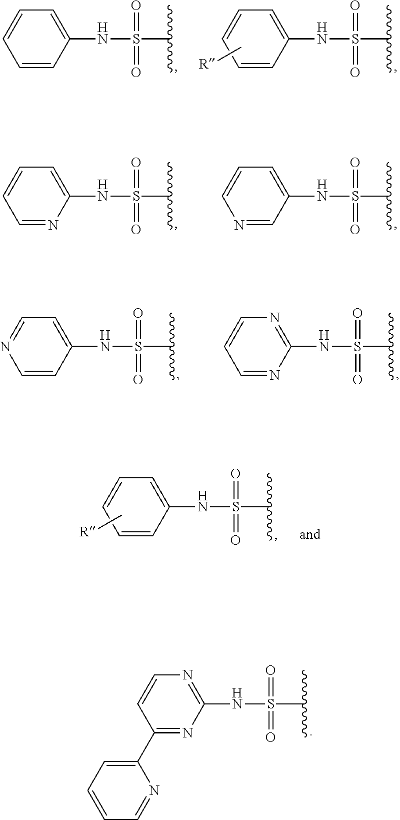

37. The method of embodiment 3, wherein R₁ is selected from the group consisting of:

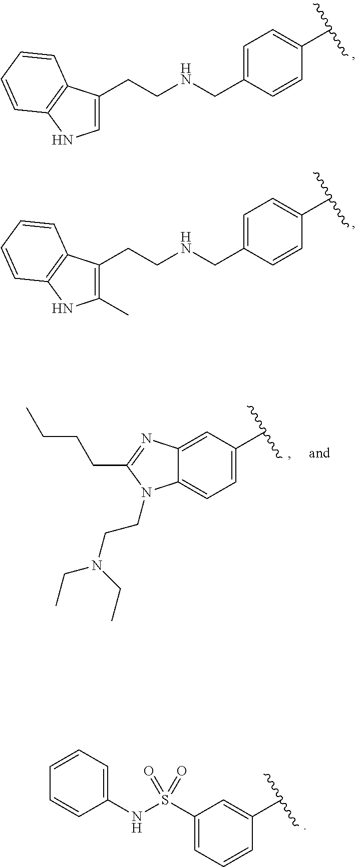 [SMILES: *c1ccc(CNCCc2c[nH]c3ccccc23)cc1 ; *c1ccc(CNCCc2c(C)[nH]c3ccccc23)cc1 ; *c1ccc2c(c1)nc(CCCC)n2CCN(CC)CC ; *c1cccc(S(=O)(=O)Nc2ccccc2)c1]

38. The method of embodiment 28, wherein n is 2.
39. The method of embodiment 38, wherein R₁ is selected from the group consisting of:

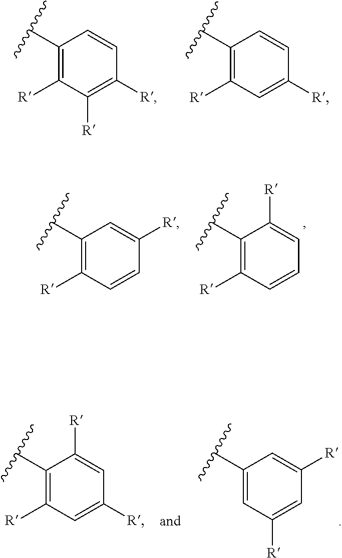

40. The method of embodiment 28, wherein n is 3.
41. The method of embodiment 40, wherein R₁ is selected from the group consisting of:

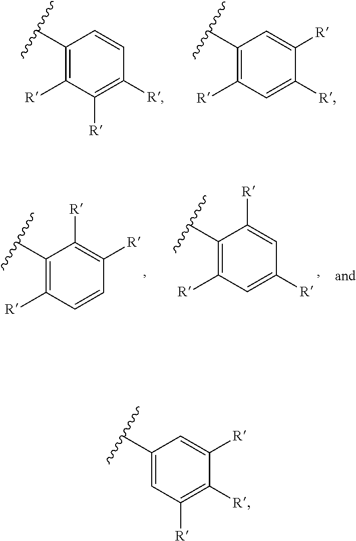

42. The method of embodiment 2, wherein R₁ is a substituted or unsubstituted heteroaryl.
43. The method of embodiment 40, wherein R₁ is an unsubstituted heteroaryl.
44. The method of embodiment 40, wherein R₁ is a substituted heteroaryl.
45. The method of embodiment 40, wherein R₁ is a N-containing heteroaryl.
46. The method of embodiment 40, wherein R₁ is a O-containing heteroaryl.
47. The method of embodiment 40, wherein R₁ is a S-containing heteroaryl.
48. The method of embodiment 40, wherein R₁ is a 5-membered heteroaryl.
49. The method of embodiment 40, wherein R₁ is a 6-membered heteroaryl.
50. The method of embodiment 40, wherein R₁ is a bicyclic heteroaryl.
51. The method of embodiment 40, wherein R₁ is a tricyclic heteroaryl.
52. The method of embodiment 2, wherein R₁ is selected from the group consisting of:

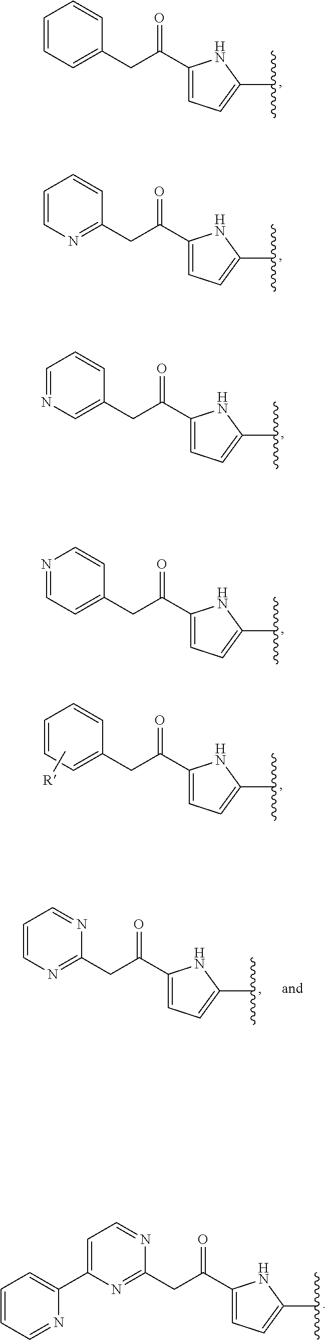

53. The method of embodiment 2, wherein R₁ is:

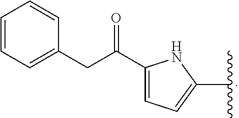 [SMILES: *c1ccc(C(=O)Cc2ccccc2)[nH]1]

54. The method of embodiment 2, wherein R₁ is-C(O) R′″, wherein R′″ is hydrogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR^{B}; —N(R^{B})₂; —NHC(O)R^{B}; or —C(R^{B})₃; wherein each occurrence of R_{B} is independently hydrogen; halogen; aliphatic; heteroaliphatic; acyl; aryl; heteroaryl; hydroxyl; alkoxy; aryloxy; alkylthioxy; arylthioxy; amino; alkylamino; dialkylamino; heteroaryloxy; or heteroarylthioxy.
55. The method of embodiment 2, wherein R₁ is selected from a group consisting of:

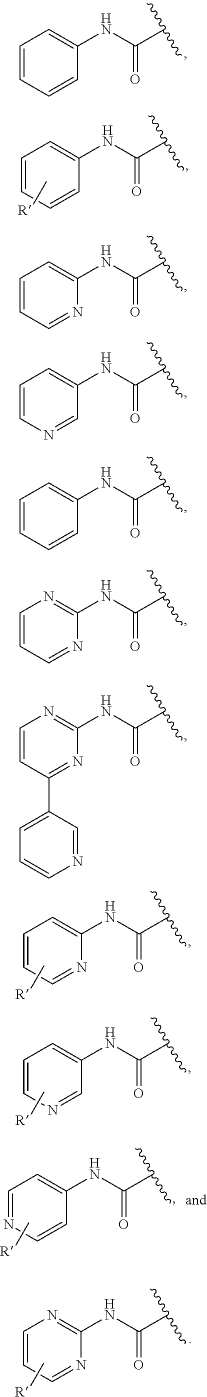

56. The method of embodiment 2, wherein R₁ is selected from the group consisting of:

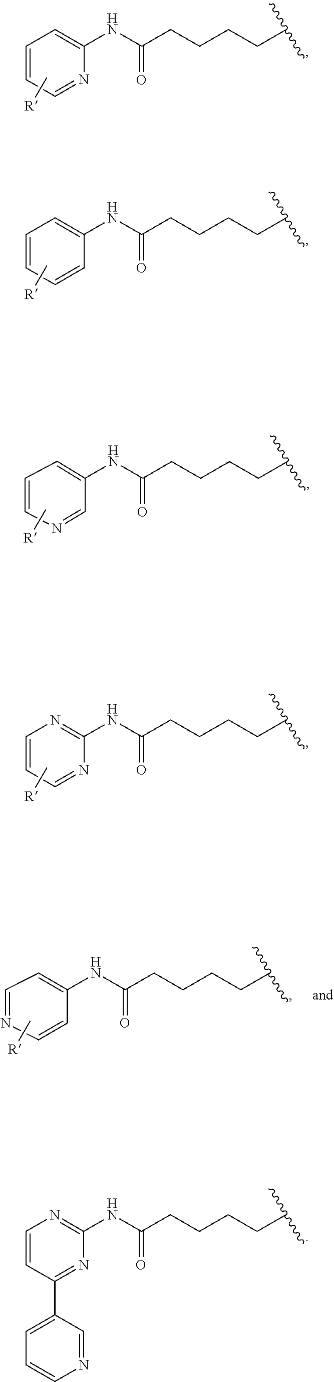

57. The method of embodiment 2, wherein R₁ is selected from the group consisting of:

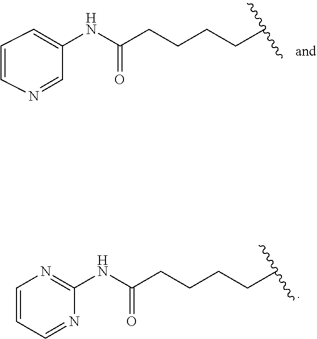 [SMILES: *CCCCC(=O)Nc1cccnc1 ; *CCCCC(=O)Nc1ncccn1]

58. The method of embodiment 22, wherein R₁ is selected from the group consisting of:

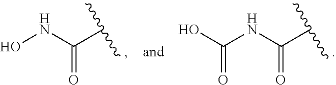 [SMILES: *C(=O)NO ; *C(=O)NC(=O)O]

59. The method of embodiment 2, wherein R₁ is

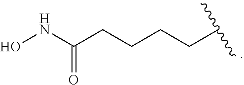 [SMILES: *CCCCC(=O)NO]

60. The method of embodiment 2, wherein R₁ is:

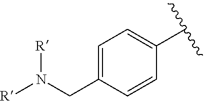

wherein each occurrence of R′ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched: unbranched heteroaryl; —OR^{A}; —C(═O)R^{A}; —CO₂R^{A}; —C(═O)N(R^{A})₂; —CN; —SCN; —SR^{A}; —SOR^{A}; —SO₂R^{A}; —NO₂; —N(R^{A})₂; —NHC(O)R^{A}; or —C(R^{A})₃; wherein each occurrence of R^{A} is independently hydrogen; halogen; aliphatic; heteroaliphatic; acyl; aryl; heteroaryl; hydroxyl; alkoxy; aryloxy; alkylthioxy; arylthioxy; amino; alkylamino; dialkylamino; heteroaryloxy; or heteroarylthioxy.
61. The method of embodiment 2, wherein R₁ is a substituted or unsubstituted heterocyclic moiety.
62. The method of embodiment 2, wherein R₁ is a monocyclic moiety.
63. The method of embodiment 2, wherein R₁ is a bicyclic moiety.
64. The method of embodiment 2, wherein R₁ is a tricyclic moiety.
65. The method of embodiment 2, wherein R₁ is selected from a group consisting of:

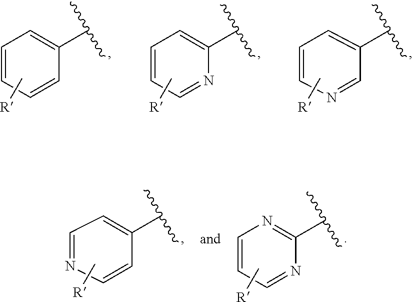

66. The method of embodiment 2, wherein R₁ is:

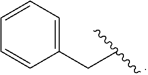 [SMILES: *Cc1ccccc1]

67. The method of embodiment 2, wherein R₁ is:

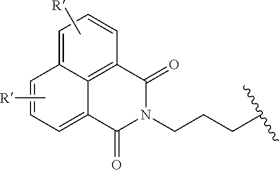

wherein R′ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR^{A}; —C(═O)R^{A}; —CO₂R^{A}; —C(═O)N(R^{A})₂; —CN; —SCN; —SR^{A}; —SOR^{A}; —SO₂R^{A}; —NO₂; —N(R^{A})₂; —NHC(O)R^{A}; or —C(R^{A})₃; wherein each occurrence of R^{A} is independently hydrogen; halogen; aliphatic; heteroaliphatic; acyl; aryl; heteroaryl; hydroxyl; alkoxy; aryloxy; alkylthioxy; arylthioxy; amino; alkylamino; dialkylamino; heteroaryloxy; or heteroarylthioxy.
68. The method of embodiment 2, wherein R₁ is:

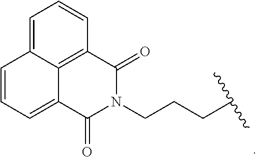 [SMILES: *CCCN1C(=O)c2cccc3cccc(c23)C1=O]

69. The method of embodiment 2, wherein R₁ is:

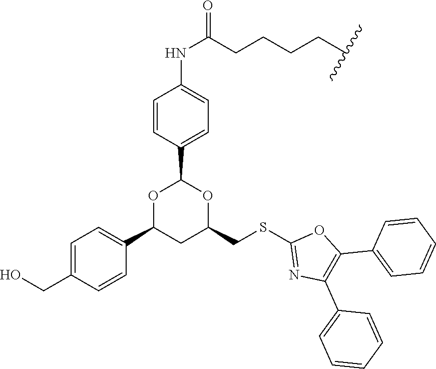 [SMILES: *CCCCC(=O)Nc1ccc([C@H]2O[C@@H](CSc3nc(-c4ccccc4)c(-c4ccccc4)o3)C[C@@H](c3ccc(CO)cc3)O2)cc1]

70. The method of embodiment 2, wherein formula (I) has the structure

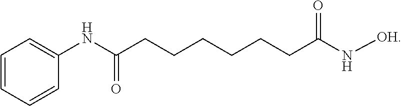 [SMILES: O=C(CCCCCCC(=O)Nc1ccccc1)NO]

71. The method of embodiment 2, wherein formula (I) has the structure

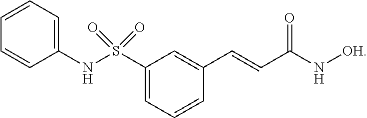 [SMILES: O=C(/C=C/c1cccc(S(=O)(=O)Nc2ccccc2)c1)NO]

72. The method of embodiment 2, wherein formula (I) has the structure

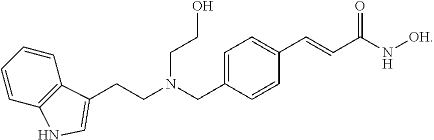 [SMILES: O=C(/C=C/c1ccc(CN(CCO)CCc2c[nH]c3ccccc23)cc1)NO]

73. The method of embodiment 2, wherein formula (I) has the structure

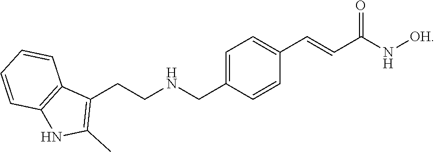 [SMILES: Cc1[nH]c2ccccc2c1CCNCc1ccc(/C=C/C(=O)NO)cc1]

74. The method of embodiment 2, wherein formula (I) has the structure

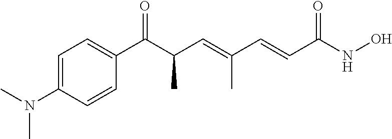 [SMILES: CC(/C=C/C(=O)NO)=C\[C@@H](C)C(=O)c1ccc(N(C)C)cc1]

75. The method of embodiment 2, wherein formula (I) has the structure

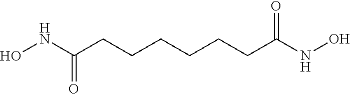 [SMILES: O=C(CCCCCCC(=O)NO)NO]

76. The method of embodiment 2, wherein formula (I) has the structure

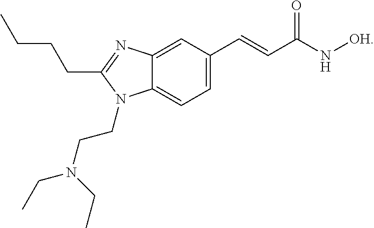 [SMILES: CCCCc1nc2cc(/C=C/C(=O)NO)ccc2n1CCN(CC)CC]

77. The method of any one of embodiments 1 to 76, wherein the hydroxamate-based histone deacetylase inhibitor is added before the stationary phase of growth of the microorganism.
78. The method of any one of embodiments 1 to 76, wherein the hydroxamate-based histone deacetylase inhibitor is added during the stationary phase of growth of the microorganism.
79. The method of embodiment 78, wherein the microorganism is cultured with the hydroxamate-based histone deacetylase inhibitor for about 0.5 to about 7 days.
80. The method of embodiment 79, wherein the microorganism is cultured with the hydroxamate-based histone deacetylase inhibitor for about 1 to about 4 days.
81. The method of embodiment 80, wherein the microorganism is cultured with the hydroxamate-based histone deacetylase inhibitor for about 3 days.
82. The method of embodiment 77, wherein the hydroxamate-based histone deacetylase inhibitor is added during the lag phase of growth of the microorganism.
83. The method of embodiment 77, wherein the hydroxamate-based histone deacetylase inhibitor is added during the exponential phase of growth of the microorganism.
84. The method of embodiments 82 or 83, wherein the microorganism is cultured with the hydroxamate-based histone deacetylase inhibitor for about 0.5 to about 14 days.
85. The method of embodiment 84, wherein the microorganism is cultured with the hydroxamate-based histone deacetylase inhibitor for about 1 to about 7 days.
86. The method of embodiment 85, wherein the microorganism is cultured with the hydroxamate-based histone deacetylase inhibitor for about 6 days.
87. The method of any one of embodiments 1 to 86, wherein the hydroxamate-based histone deacetylase inhibitor is added at a concentration of 0.001-100 μM.
88. The method of embodiment 87, wherein the hydroxamate-based histone deacetylase inhibitor is added at a concentration of 0.01-100 μM.
89. The method of embodiment 88, wherein the hydroxamate-based histone deacetylase inhibitor is added at a concentration of 0.1-80 μM.
90. The method of embodiment 89, wherein the hydroxamate-based histone deacetylase inhibitor is added at a concentration of 1-40 μM.
91. The method of embodiment 90, wherein the hydroxamate-based histone deacetylase inhibitor is added at a concentration of 2-20 μM.
92. The method of embodiment 91, wherein the hydroxamate-based histone deacetylase inhibitor is added at a concentration of 2.5-10 μM.
93. The method of embodiment 92, wherein the hydroxamate-based deacetylase inhibit is added at a concentration of 2.5, 5 or 10 μM.
94. The method of embodiment 93, wherein the hydroxamate-based deacetylase inhibit is added at a concentration of 5 μM.
95. The method of any one of embodiments 1 to 94, wherein cell growth kinetics and/or cell viability of the microorganism is at least 60% of a control.
96. The method of embodiment 95, wherein cell growth kinetics and/or cell viability of the microorganism is at least 70% of a control.
97. The method of embodiment 95, wherein cell growth kinetics and/or cell viability of the microorganism is at least 80% of a control.
98. The method of embodiment 95, wherein cell growth kinetics and/or cell viability of the microorganism is at least 90% of a control.
99. The method of embodiment 95, wherein cell growth kinetics and/or cell viability of the microorganism is at least 95% of a control.
100. The method of embodiment 95, wherein cell growth kinetics and/or cell viability of the microorganism is at least 99% of a control.
101. The method of any one of embodiments 95 to 100, wherein cell growth kinetics is determined by optical density.
102. The method of any one of embodiments 95 to 100, wherein cell viability is determined by membrane integrity confirmation using propidium iodide staining.
103. The method of any one of embodiments 1 to 102, wherein the microorganism was stored before culturing.
104. The method of embodiment 103, wherein the microorganism was stored on or in a culture or a storage medium.
105. The method of embodiment 103, wherein the microorganism was stored by cryopreservation.
106. The method of embodiment 103, wherein the microorganism was stored by lyophilization.
107. The method of any one of embodiments 103 to 106, wherein the microorganism has been stored for at least a day.
108. The method of embodiment 107, wherein the microorganism has been stored for at least a week.
109. The method of embodiment 108, wherein the microorganism has been stored for at least 1 month.
110. The method of 109, wherein the microorganism has been stored for at least 3 months.
111. The method of 110, wherein the microorganism has been stored for at least 6 months.
112. The method of 111, wherein the microorganism has been stored for at least 1 year.
113. The method of any one of embodiments 1 to 112, wherein the microorganism is a microalga, stramenophile, protist or fungus.
114. The method of embodiment 113, wherein the microalga is a species from Acutodesmus, Ankistrodesmus, Asteromonas, Aurantiochytrium, Auxenochlorella, Basichlamys, Botryococcus, Botryokoryne, Borodinella, Brachiomonas, Catena, Carteria, Chaetophora, Characiochloris, Characiosiphon, Chlainomonas, Chlamydomonas, Chlorella, Chlorochytrium, Chlorococcum, Chlorogonium, Chloromonas, Closteriopsis, Dictyochloropsis, Dunaliella, Ellipsoidon, Eremosphaera, Eudorina, Floydiella, Friedmania, Haematococcus, Hafniomonas, Heterochlorella, Gonium, Halosarcinochlamys, Koliella, Lobocharacium, Lobochlamys, Lobomonas, Lobosphaera, Lobosphaeropsis, Marvania, Monoraphidium, Myrmecia, Nannochloris, Nannochloropsis, Oocystis, Oogamochlamys, Pabia, Pandorina, Parietochloris, Phacotus, Phaeodactylum, Platydorina, Platymonas, Pleodorina, Polulichloris, Polytoma, Polytomella, Prasiola, Prasiolopsis, Prasiococcus, Prototheca, Pseudochlorella, Pseudocarteria, Pseudotrebouxia, Pteromonas, Pyrobotrys, Rosenvingiella, Scenedesmus, Schizochytrium, Spirogyra, Stephanosphaera, Tetrabaena, Tetraedron, Tetraselmis, Thalassiosira, Thraustochytium, Trebouxia, Trochisciopsis, Ulkenia, Viridiella, Vitreochlamys, Volvox, Volvulina, Vulcanochloris, Watanabea, Yamagishiella, Euglena, Isochrysis, or Nannochloropsis.
115. The method of embodiment 114, wherein the microalga is Chlorella vulgaris, Chlorella sorokiniana, Chlorella protothecoides, Chlamydomonas reinhardtii or Schizochytrium limacinum.
116. The method of embodiment 113, wherein the microalga is a species of the class Chlorophyceae.
117. The method of embodiment 116, wherein the microalga is a species of the order Chlamydomonadales.
118. The method of embodiment 117, wherein the microalga is a species of the family Chlamydomonadaceae.
119. The method of embodiment 118, wherein the microalga is a species of the genus Chlamydomonas.
120. The method of embodiment 119, wherein the microalga is Chlamydomonas reinhardtii.
121. The method of embodiment 113, wherein the microalga is a diatom or stramenophile.
122. The method of embodiment 121, wherein the microalga is Phaeodactylum tricornutum or Thalassiosira pseudonana.
123. The method of embodiment 122, wherein the microalga is Phaeodactylum tricornutum.
124. The method of any one of embodiments 1 to 123, wherein the exogenous nucleic acid encodes a protein of interest.
125. The method of embodiment 124, wherein the protein of interest is an enzyme, antibody, DNA-binding protein, transcription regulating protein, membrane protein, nuclear receptor protein, nuclear localization signal protein, protein cofactor, intracellular structural protein, or a fragment or derivative thereof.
126. The method of embodiment 125, wherein the enzyme is a biosynthetic enzyme.
127. The method of embodiment 126, wherein the biosynthetic enzyme is an oxidoreductase, transferase, hydrolase, lyase, isomerase, ligase, or translocase.
128. The method of any one of embodiments 1 to 123, wherein the exogenous nucleic acid encodes an RNA molecule.
129. The method of embodiment 128, wherein the RNA molecule is an miRNA, siRNA, shRNA, or a DsiRNA.

The invention will now be described by way of non-limiting examples.

### Example 1

### Methods

Chlamydomonas reinhardtii Cultivation and Transformation

Chlamydomonas reinhardtii strain CC-1690 (wild-type strain; WT) was purchased from the Chlamydomonas Resource Center (The University of Minnesota, USA). Growth of C. reinhardtii was performed under mixotrophic conditions with Tris acetate phosphate (TAP) medium (Gorman & Levine, 1965) on agar plates or liquid in 6 well plates, 96 well plates or 125 mL shake flasks under 50±10 μmol photons m⁻² s⁻¹ light intensity with alternating light: dark cycles (16:8) at 21±0.5° C.

Treatment with Histone Deacetylase Inhibitors

For all assays, colonies were initially transferred from agar plates to a liquid preculture in 6 well plates for 4-6 days, with the above-mentioned culture conditions.

For the sirtinol, SAHA, OSS-128167, nicotinamide and sodium butyrate series of treatments (comparison between various HDAC inhibitors), inoculums of 10⁵ cells in late exponential phase of growth were transferred in 200 μL of TAP medium in 96 well plates and were treated with 50 μM, 2.5 μM, 100 μM, 1 mM and 5 mM of each histone deacetylase inhibitor respectively for 24 hours; a mix of all inhibitors was also tested. DMSO was used as a negative control.

For the SAHA (vorinostat), belinostat, dacinostat, panobinostat, mocetinostat, entinostat, and romidepsin series of treatments (comparison between various SAHA analogs and other HDAC inhibitors), inoculums of 10⁵ cells in late exponential phase of growth were transferred in 200 μL of TAP medium in 96 well plates and were treated with 5 μM of each histone deacetylase inhibitor for 6 days. DMSO was used as a negative control.

For the SAHA main treatments, inoculum of 10⁵ cells in late exponential phase of growth were transferred in 50 mL of TAP medium in 125 mL shake flasks and were treated with 5 μM for 12 days (to evaluate mVenus expression overtime), or with 2.5, 5 and 10 μM for 6 days (to evaluate SAHA toxicity over cell viability). DMSO was used as a negative control.

Growth Curve, Chlorophyll and mVenus Detection with Plate Reader

Growth curves were obtained using a Synergy H1 plate reader (Biotek, Agilent), from two or three biological replicates for each strain studied as in (Molino et al., 2022). For SAHA main treatments, 250 μL samples of 50 mL cultures were added in 96 well flat bottom microplates (Corning Costar 96-Well, Cell Culture-Treated, Fisher Scientifics). The OD absorbance was read at 750 nm. Bottom read scan mode mean fluorescence was measured with Excitation (Ex): 500 nm/Emission (Em): 541 nm for mVenus; and bottom read fluorescence Ex: 475 nm/Em: 650 nm for chlorophyll.

Flow Cytometry mVenus Detection by FACS for Screening

A Cytomics FC500 cytometer equipped with Argon (488 nm) and HeNe (633 nm) lasers were used to measure mVenus emission on the FL1 channel (525/15 nm), and chloroplast fluorescence on the FL4 channel (675/15 nm) (Beckman Coulter Life Sciences). At least 10 000 events were acquired. A homogeneous cloud of intact cells was first gated based on size (FSC) and granulosity (SSC). Then a daughter gate selected the cloud of cells with homogenous chlorophyll autofluorescence. The % of mVenus⁺ cells was measured on this daughter population with a gate in the FL1 channel that excluded cells with autofluorescence levels similar to the negative controls. Cells were considered mVenus⁺ when both the % of gated mVenus⁺ cells (and >0.1%) and mVenus mean fluorescence intensity were higher than the values of the WT controls. Propidium iodide (Thermo Fisher) was used to verify viability and acquired on the FL3 channel (620/20 nm). To select for palmelloid cells, a second gate that included events with a minimum of 2-fold increase in FSC mean intensity was drawn, similarly to (Cheloni and Slaveykova, 2021). In that case, cells were acquired on a Beckman Cytoflex S equipped with violet (405 nm), blue (488 nm), yellow-green (561 nm) and red (638 nm) lasers. Chlorophyll autofluorescence was detected in the PerCP channel (690/50 nm), mVenus on the FITC (525/40BP). Statistics were obtained using BD FlowJo version 10 software (BD Biosciences, La Jolla, CA, USA, 2020).

RNA Extraction and RT-qPCR for mRNA Relative Quantity

4 mL of a 6 days C. reinhardtii grown culture were harvested and centrifuged in Eppendorf tube for 5 min at 4000 g. Cells were lysed by immersion in liquid nitrogen for 1 min. Total RNA was extracted using Invitrogen™ TRIzol™ reagent (Life technologies, Thermo Fisher Scientifics) according to the manufacturer's protocol with minor modifications, including the addition of NaCl at a final concentration of 100 mM in the isopropanol at the nucleic acids precipitation step. Samples were further treated with Turbo DNase (Invitrogen™ TURBO DNA-free™ Kit, Thermo Fisher Scientifics) according to the manufacturer's instructions. RNA yield and quality were determined with 260 nm/280 nm absorbance ratio obtained on a Nanodrop instrument (Thermo Fisher Scientific). Samples of <100 ng total RNA were subjected to reverse transcription and qPCR amplification in a single reaction using the Luna® Universal One-Step RT-qPCR Kit Protocol (New England Biolabs Canada). Briefly, 1 μl of RNA were subjected to reverse transcription performed at 55° C. for 10 minutes. Initial denaturation was 1 min at 95° C. followed by 45 cycles of denaturation for 10 seconds, extension for 30 seconds at 60° C. A melt curve analysis was performed from 60-95° C. with an increment of 0.5° C. each 5 seconds. Primers used for the mVenus transcript and histone H3 housekeeping gene (Liu et al., 2012) were designed using PrimerQuest™ Tool (Integrated DNA Technologies, IDT). SYBR Green fluorescence was recorded in the FAM channel of a CFX connect real time system (BioRad). Experiments were performed using three technical replicates from one biological template. Relative mRNA expression levels were determined according to the 2^{(-ddCt)} method.

### Statistical Analysis

Statistical analyses were performed using GraphPad Prism (Version 9.4.1, GraphPad Software, US). Data are expressed as means±SD of two or three biological replicated performed at least twice in independent experiments. Parametric tests (ANOVA and Student's t test) were used when population followed normal distribution, and non-parametric (ANOVA and Mann-Whitney test) were used when the population could not be assumed to be normally distributed. A p value <0.05 was considered to be significant.

### Results

Screening of mVenus+ Transformants by Flow Cytometry

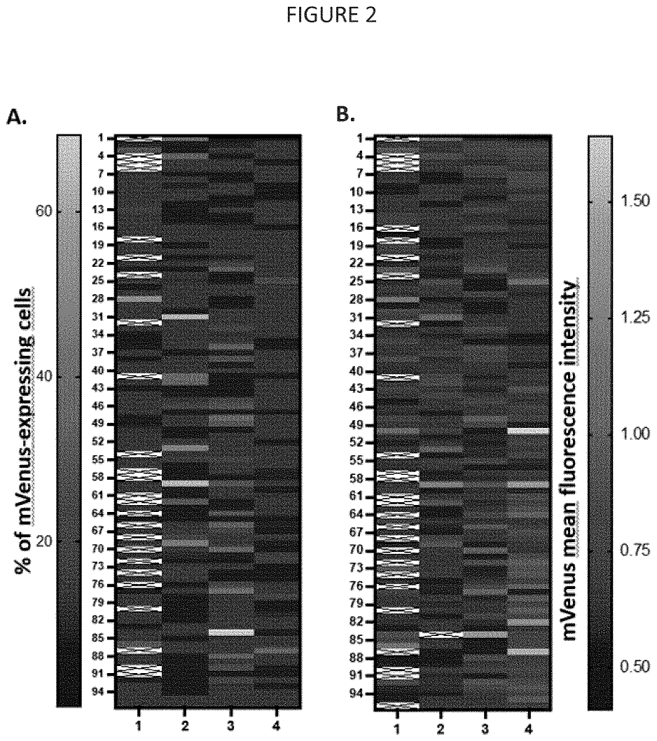

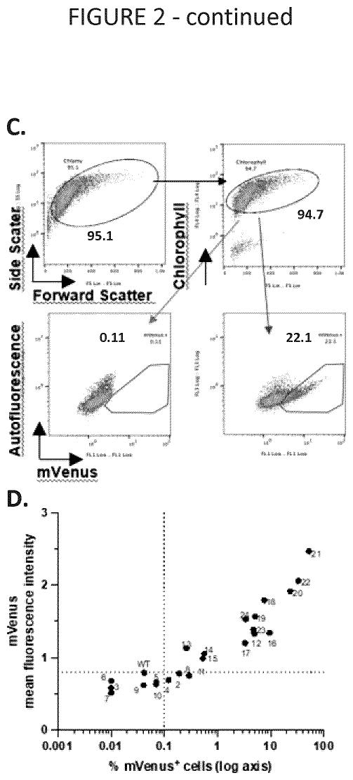

Electroporated cells with pOpt_mVenus_Paro vector were allowed to recover in liquid media for 24 hours and plated on Petri dishes containing TAP solid medium+paromomycin for 5-7 days. Cells were replated on new dishes 2 times before being cultured in liquid media for 4-6 days in 6 well plates or 96 well plates. A total of 88 (22.9%) cultures over the 384 antibiotic-resistant colonies selected presented >1% of mVenus+ cells and a mean fluorescent intensity (MFI) of acquired event above background levels of WT cells at day 6 of culture (FIGS. 2A and 2B). Of these positive cultures, 23 were selected and tested again 6 weeks later; 10 (43.5%) were still mVenus+ (>1% mVenus+ cells and mean fluorescence intensity >1.5-fold mean fluorescence intensity of WT), while the others lost detectable expression of mVenus (FIGS. 2C and 2D).

mVenus Expression Levels is Modulated by Hydroxamate Family of HDAC Inhibitors

Transformants (clones 16, 17, 18, and 20) were selected to assess if chemical treatment with silencing inhibitors could increase mVenus expression. Sirtinol, a HDAC class III/sirtuin inhibitor; SAHA, a HDAC class I, II & IV inhibitor; sodium butyrate, a HDAC class I & IIA inhibitor; nicotinamide, a HDAC class III/sirtuin inhibitor, and OSS-128167 a HDAC class III/sirtuin inhibitor specific for sirtuin 6 (Wang & al., 2019) were used, all at initiation of culture.

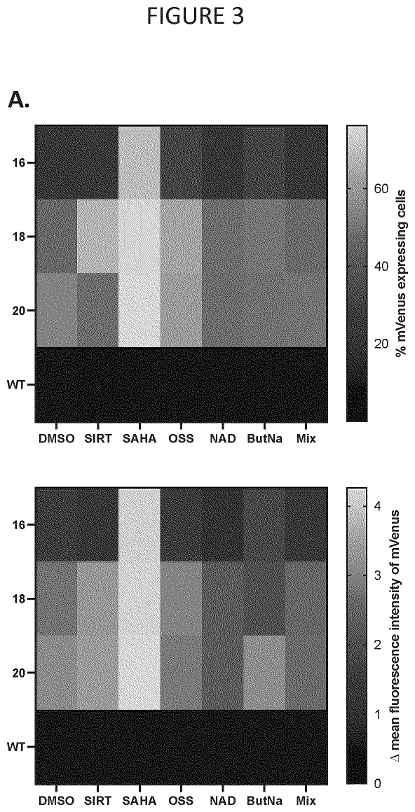

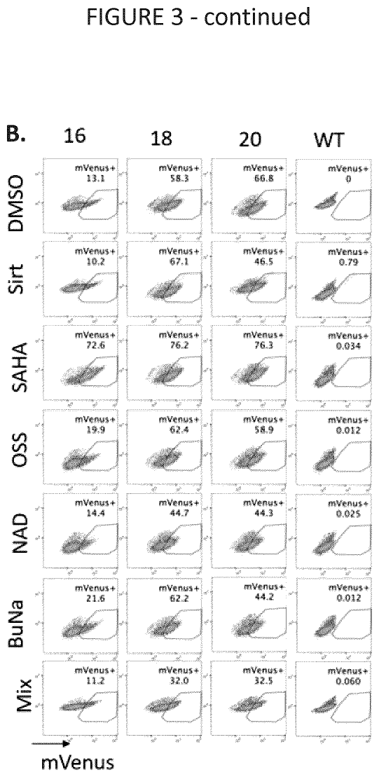

Treatment with SAHA (2.5 μM) for 24 hours increased the frequency of cells expressing mVenus and increased the level of expression of mVenus per cell (FIGS. 3A and 3B).

The % of mVenus cells increased 5.1-fold in clone 16 (13.3 to 68.1%) and there was a 4.6-fold increase in protein expression (mean fluorescence intensity) when cells were treated with SAHA. None of the other inhibitor consistently improved expression levels of mVenus.

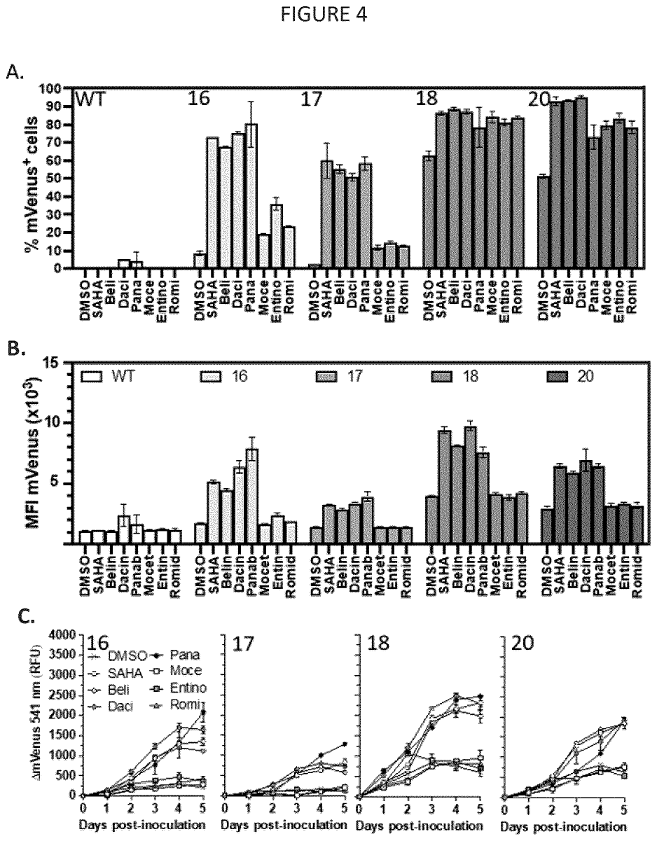

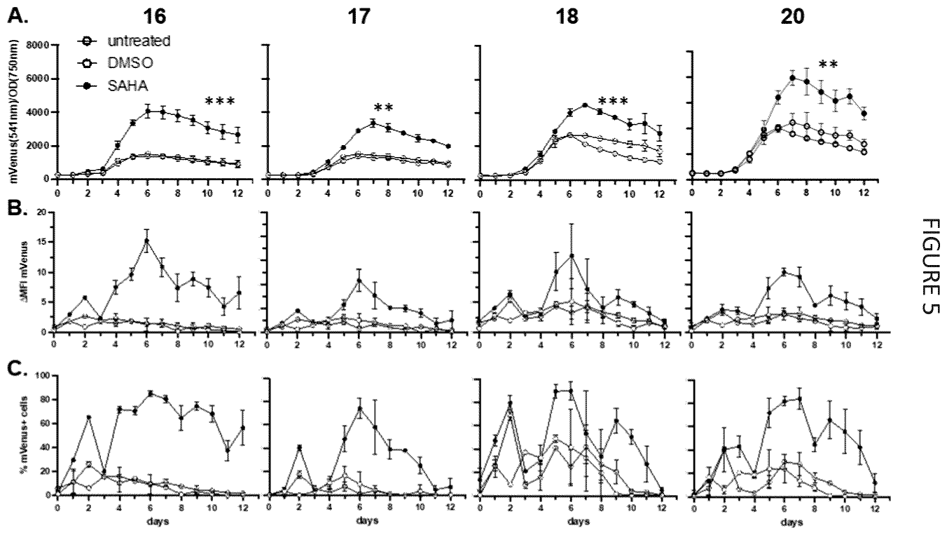

A 6-day treatment of SAHA and new generation HDAC inhibitors, i.e. Belinostat, Dacinostat and Panobinostat of hydroxamate-class like SAHA (HDAC class I, II & IV inhibitor), Mocetinostat and Entinostat, two benzamide derivatives (HDAC class I inhibitor), and Romidepsin, a bacterial-derived cyclic tetrapeptide (HDAC class I inhibitor) (Karagiannis & Rampias, 2021) was evaluated, all at 5 μM at initiation of culture (FIG. 4).

The hydroxamate class of HDACs inhibitors was the most potent in boosting both the frequency of mVenus-positive cells in silenced clones (8.6-fold in clone 16 for SAHA (8.5 to 73.1%), 27-fold in clone 17 for SAHA (2.2 to 59.9%)) and the amount of mVenus inside each cell (mean fluorescence intensity, 3-4-fold in clone 16 for SAHA, 2-5-fold in clone 17) at day 3 post-treatment (FIG. 4). Results relating to expression level were comparable between the hydroxamate-based inhibitors of this class.

mVenus Levels are Modulated Over Time and Treatment

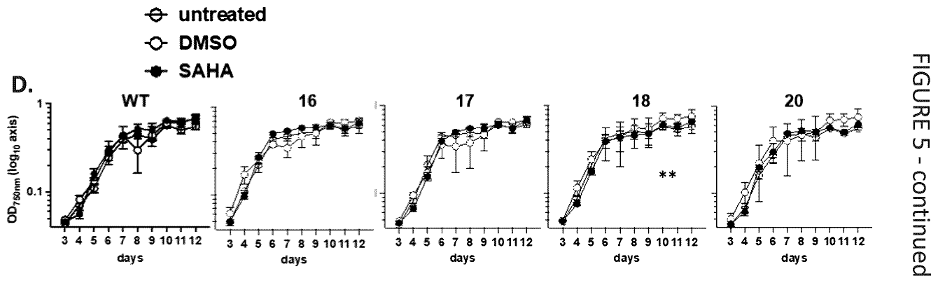

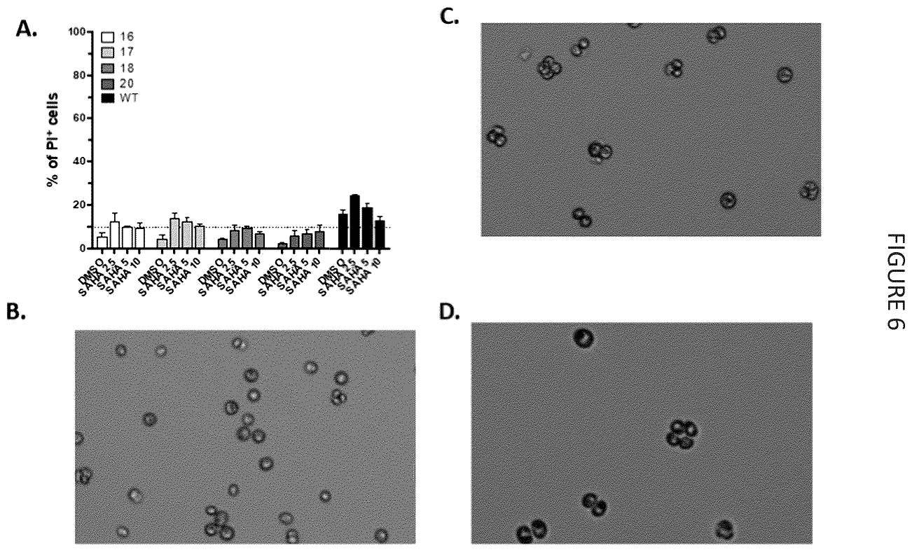

mVenus expression was followed using both a microplate reader and a flow cytometer for 12 days after treatment with SAHA. In untreated, or solvent-treated cells, overall fluorescence peaked around day 6 in all samples (day 5-7), corresponding to the end of the exponential phase and the start of the stationary phase (FIG. 5). Fluorescence levels of mVenus progressively decreased over the following 6 days of culture. When cells were treated with SAHA, the maximum production of mVenus was also measured at the end of the exponential phase (day 5-7), reaching more than 74% of producing cells in all the clones (FIG. 5C), and a maximum of fluorescence intensity overall and per cell (FIG. 5A and FIG. 5B). Consistently with the low number of cells impacting on the flow cytometer sensitivity in the first 3 days, the analysis of the % of mVenus+ cells varied a lot at early time points (FIG. 5C).

Nonetheless, SAHA treatment at day 0 of culture steadily increased fluorescence around day 4-5 in all clones, but did not prevent the progressive decline in production when transformants advanced into stationary phase around day 7-8. At day 12, most clones had only <10% or mVenus+ cells (except 16) but had still more frequent population of mVenus expressing cells and more mVenus fluorescence per cells compared to untreated or DMSO treated cells (FIG. 5). SAHA did not impact the overall growth of microalgae (FIG. 5D).

### SAHA's Effect on Viability, Cell Growth, Palmelloid and Chlorophyll Levels

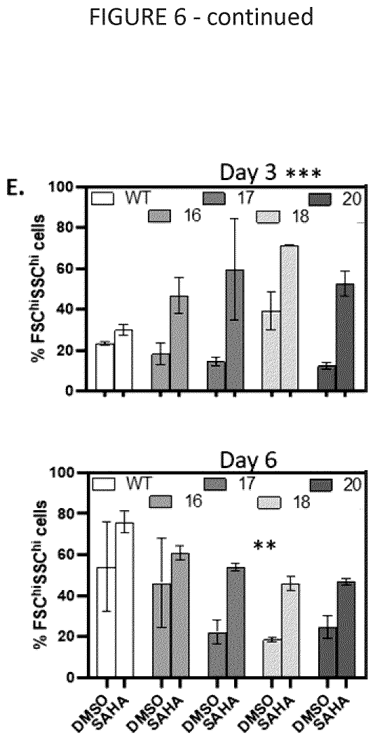

Cells were treated with increasing concentrations of the inhibitors for 6 days. Viability was analyzed through membrane integrity confirmation using propidium iodide (PI) staining at day 6 on a flow cytometer. SAHA's addition at concentration of 2.5, 5 and 10 μM slightly increased the % of PI+ cells, but ~90% of the transformant cells remained intact and there was no significant increase compared to background levels in WT cells (FIG. 6A). Different stress conditions have been reported to transiently induce palmelloid colonies in C. reinhardtii without impacting viability (de Carpentier et al., 2019). It was observed that 6 days of treatment with low doses (5 and 10 μM) of SAHA lead to the accumulation of small palmelloid colonies in C. reinhardtii (FIGS. 6C and 6D compared to FIG. 6B), while motility was also progressively lost above 2.5 μM. Palmelloid colony formation was quantified by flow cytometry when transformants were treated with 5 μM of SAHA. In most cases palmelloid colonies increased at day 3 and 6 post-treatment (FIG. 6E).

SAHA's addition at higher concentration of 20, 40 and 80 μM was also tested to see at which degree it could affect the growth kinetic and viability (FIG. 6F). Larger palmelloid colonies were observed with increasing concentrations, while growth kinetic was inhibited partially with 20 and 40 μM SAHA, but totally inhibited with 80 μM SAHA. Chlorophyll content was also strongly modified, as observed by the progressive loss of the usual dark green color of C. reinhardtii healthy culture in favor of a more yellow tint (20 and 40 μM SAHA) or even red tint (80 UM SAHA).

SAHA Upregulates mRNA Transgene Levels by Inhibiting Histone Deacetylation.

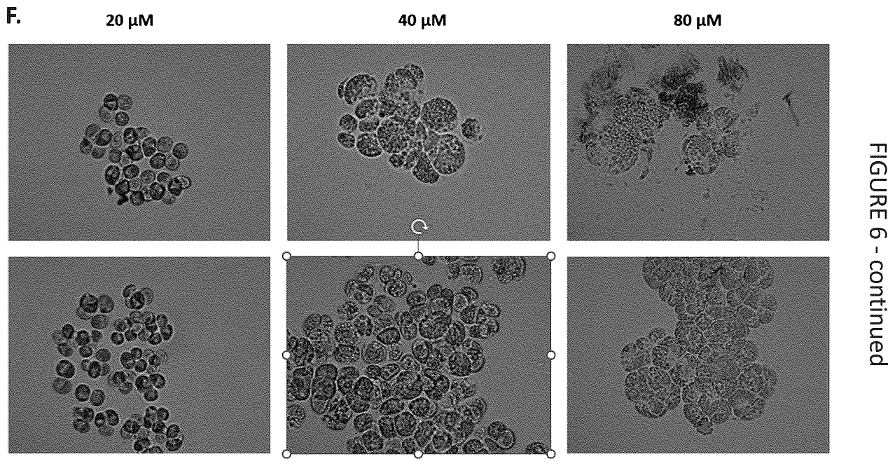

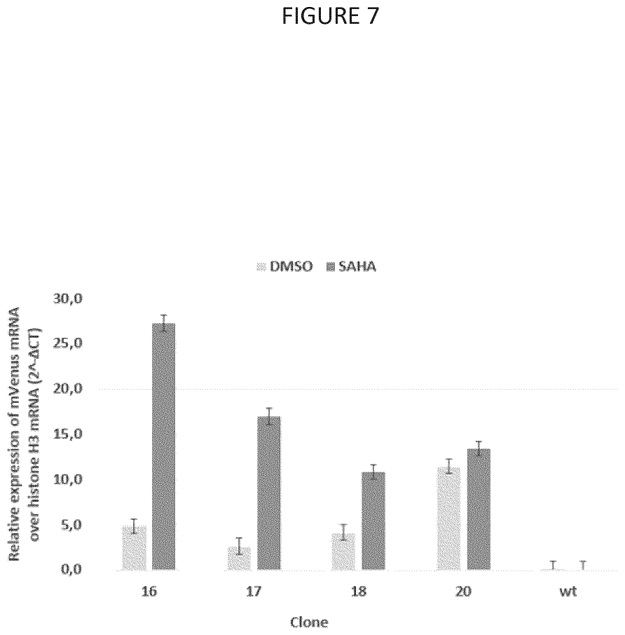

The impact of SAHA's treatment on transgene mRNA levels was measured. The relative mVenus mRNA expression over the housekeeping gene histone H3 mRNA expression increased 7-fold in clone 16 and 6.6-fold in clone 17 at day 6 of cultures when cells were treated with SAHA (FIG. 7).

### Example 2

### Method

Treatment with Histone Deacetylase Inhibitors

Inoculums of 10⁵ cells in late exponential phase of growth were transferred in 200 μL of TAP medium in 96 well plates and were treated with 50 μM of sirtinol, 5 μM of SAHA, 1 μM of azacytidine, 100 μM of OSS_128167, 1 mM of cinnamic acid, 2.5 μM of lycorine, or 5 mM of sodium butyrate as histone deacetylase inhibitor for 72 hours. DMSO was used as a negative control.

The cultivation and FACS conditions used are described above.

### Results

mVenus Levels were Modulated by Hydroxamate Family of HDAC Inhibitors Following Storage of Cells for One Year

Transformants (clones 16, 17, 18, and 20) and wild-type (WT) Chlamydomonas reinhardtii were stored for one year after transformation. The transformants were tested to assess whether treatment with histone deacetylase inhibitors could increase mVenus expression. Sodium butyrate, cinnamic acid, lycorine, OSS-128167, Sirtinol, SAHA and azacitidine were used, all at the initiation of culture.

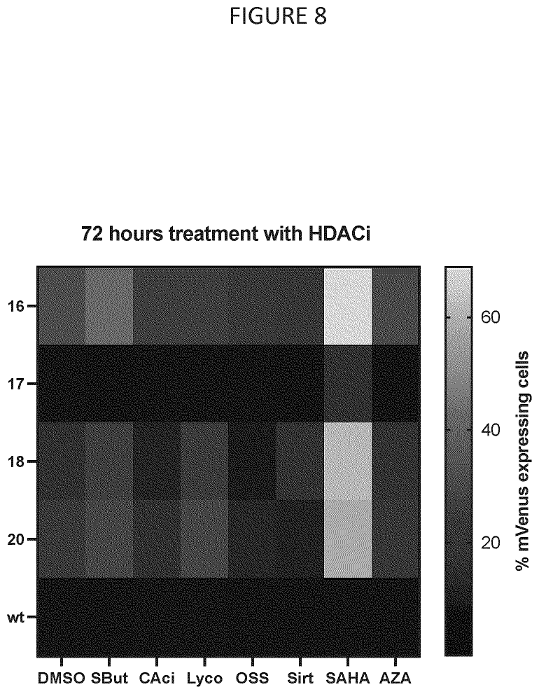

Treatment with SAHA (5 μM) for 72 hours increased the frequency of cells expressing mVenus (FIG. 8).

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

All publications and patents cited herein are incorporated by reference in their entirety as if each individual publication or patent were specifically and individually indicated to be incorporated by reference. In the case of any conflict between a definition of a term in the present disclosure and a definition in a cited publication or patent, the definition provided in the present disclosure is to be used in describing the present invention.

### REFERENCES

Baier, T., Wichmann, J., Kruse, O., and Lauersen, K. J. (2018). Intron-containing algal transgenes mediate efficient recombinant gene expression in the green microalga Chlamydomonas reinhardtii. Nucleic Acids Res 46 (13), 6909-6919. doi: 10.1093/nar/gky532.
Casas-Mollano, J. A., van Dijk, K., Eisenhart, J., and Cerutti, H. (2007). SET3p monomethylates histone H3 on lysine 9 and is required for the silencing of tandemly repeated transgenes in Chlamydomonas. Nucleic Acids Res 35 (3), 939-950. doi: 10.1093/nar/gkl1149.
Cecchin, M., Benfatto, S., Griggio, F., Mori, A., Cazzaniga, S., Vitulo, N., Delledonne, M., Ballottari, M. (2018). Molecular basis of autotrophic vs mixotrophic growth in Chlorella sorokiniana. Sci Rep, 8 (1), 6465. doi: 10.1038/s41598-018-24979-8.
Cheloni, G., and Slaveykova, V. I. (2021). Morphological plasticity in Chlamydomonas reinhardtii and acclimation to micropollutant stress. Aquat Toxicol 231, 105711. doi: 10.1016/j.aquatox.2020.105711.
de Carpentier, F., Lemaire, S. D., and Danon, A. (2019). When Unity Is Strength: The Strategies Used by Chlamydomonas to Survive Environmental Stresses. Cells 8 (11), 1307. doi: 10.3390/cells8111307.
de Carvalho, J. C., Sydney, E. B., Tessari, L. F., and Soccol, C. R. (2019) Biofuels from Algae 2^{nd} Ed., Elsevier, 2019, 33-50. ISBN 9780444641922.
Gorman, D. S., and Levine, R. P. (1965). Cytochrome f and plastocyanin: their sequence in the photosynthetic electron transport chain of Chlamydomonas reinhardtii. Proc. Natl. Acad. Sci. 54 (6), 1665-1669. doi: 10.1073/pnas.54.6.1965.
Hassig, C. A. and Schreiber, S. L. (1997). Nuclear histone acetylases and deacetylases and transcriptional regulation: HATs off to HDACs. Curr. Opin. Chem. Biol 1 (3), 300-308. doi: 10.1016/S1367-5931 (97) 80066-X
Kaginkar, S., Priya, S., Sharma, U., D'Souza, J. S., and Sen, S. (2021). A potential screening method for epigenetic drugs: uncovering stress-induced gene silencing in Chlamydomonas. Free Radic Res 55 (5), 533-546. doi: 10.1080/10715762.2021.1876231.
Karagiannis, D. and Rampias, T. (2021). HDAC Inhibitors: Dissecting Mechanisms of Action to Counter Tumor Heterogeneity. Cancer 13 (14), 3575-3603. doi: 10.3390/cancers13143575
Kurniasih, S. D., Yamasaki, T., Kong, F., Okada, S., Widyaningrum, D., and Ohama, T. (2016). UV-mediated Chlamydomonas mutants with enhanced nuclear transgene expression by disruption of DNA methylation-dependent and independent silencing systems. Plant Mol Biol 92 (6), 629-641. doi: 10.1007/s11103-016-0529-9.
Liu, C., Wu, G., Huang, X., Liu, S., and Cong, B. (2012). Validation of housekeeping genes for gene expression studies in an ice alga Chlamydomonas during freezing acclimation. Extremophiles 16 (3), 419-425. doi: 10.1007/s00792-012-0441-4.

Molino, J. V. D., Carpine, R., Gademann, K., Mayfield, S., and Sieber, S. (2022). Development of a cell surface display system in Chlamydomonas reinhardtii. Algal Research 61, 102570. doi: https://doi.org/10.1016/j.algal.2021.102570.
Neupert, J., Gallaher, S. D., Lu, Y., Strenkert, D., Segal, N., Barahimipour, R., et al. (2020). An epigenetic gene silencing pathway selectively acting on transgenic DNA in the green alga Chlamydomonas. Nat Commun 11 (1), 6269. doi: 10.1038/s41467-020-19983-4.
Neupert, J., Karcher, D., and Bock, R. (2009). Generation of Chlamydomonas strains that efficiently express nuclear transgenes. Plant J 57 (6), 1140-1150. doi: 10.1111/j.1365-313X.2008.03746.x.
Pandey, A., Lee, D. J., Chisti, Y., and Soccol, C. (2013). Biofuels from Algae 1^{st} Ed., Elsevier, ISBN 9780444595584.
Schroda, M., Blocker, D., and Beck, C. F. (2000). The HSP70A promoter as a tool for the improved expression of transgenes in Chlamydomonas. Plant J 21 (2), 121-131. doi: 10.1046/j.1365-313x.2000.00652.x.
Singh, S. P., and Singh, P. (2014). Effect of CO2 concentration on algal growth: A review. Ren Sus En Rev, 38, 172-179. doi: 10.1016/j.rser.2014.05.043
Shaver, S., Casas-Mollano, J. A., Cerny, R. L., and Cerutti, H. (2010). Origin of the polycomb repressive complex 2 and gene silencing by an E (z) homolog in the unicellular alga Chlamydomonas. Epigenetics 5 (4), 301-312. doi: 10.4161/epi.5.4.11608.
Specht, E. A., Nour-Eldin, H. H., Hoang, K. T., and Mayfield, S. P. (2015). An improved ARS2-derived nuclear reporter enhances the efficiency and ease of genetic engineering in Chlamydomonas. Biotechnol J 10 (3), 473-479. doi: 10.1002/biot.201400172.
Wang, Y., He, J., Liao, M., Hu, M., Li, W., Ouyang, H., Wang, X., Ye, T., Zhang, Y., Ouyang, L. (2019). An overview of Sirtuins as potential therapeutic target: Structure, function and modulators. Eur J Med Chem 161, 48-77, doi: 10.1016/j.ejmech.2018.10.028
Waterborg, J. H., Robertson, A. J., Tatar, D. L., Borza, C. M., and Davie, J. R. (1995). Histones of Chlamydomonas reinhardtii. Synthesis, acetylation, and methylation. Plant Physiol 109 (2), 393-407. doi: 10.1104/pp. 109.2.393.

1-38. (canceled)

39. A method of increasing expression of an exogenous nucleic acid in a microorganism comprising adding a hydroxamate-based histone deacetylase inhibitor to a culture medium of the microorganism.

40. The method of claim 39, wherein the hydroxamate-based deacetylase inhibitor has the structure of: 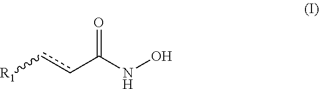
wherein
R₁ is a cyclic or acyclic, substituted or unsubstituted, branched or unbranched aliphatic; a cyclic or acyclic, substituted or unsubstituted, branched or unbranched heteroaliphatic; a substituted or unsubstituted aryl, or a substituted or unsubstituted heteroaryl; and [symbol]
represents a single or double bond.

41. The method of claim 40, wherein R₁ is selected from the group consisting of: 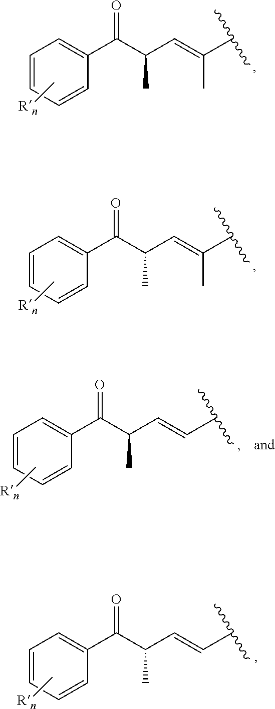
wherein
n is an integer between 0 and 5, inclusive; and
each occurrence of R′ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branded or unbranched acyl; substituted or unsubstituted, branched or unbranched aryl; substituted or unsubstituted, branched or unbranched heteroaryl; —OR^{B}; —C(═O)R^{B}; —CO₂R^{B}; —C(═O)N(R^{B})₂; —CN; —SCN; —SR^{B}; —SOR^{B}; —SO₂R^{B}; —NO₂; —N(R^{B})₂; —NHC(O)R^{B}; or —C(R^{B})₃; wherein each occurrence of R_{B} is independently hydrogen; halogen; aliphatic; heteroaliphatic; acyl; aryl; heteroaryl; hydroxyl; alkoxy; aryloxy; alkylthioxy; arylthioxy; amino; alkylamino; dialkylamino; heteroaryloxy; or heteroarylthioxy.

42. The method of claim 40, wherein R₁ is selected from the group consisting of: 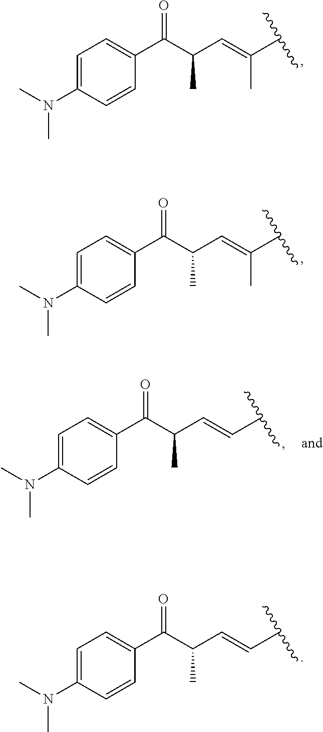 [SMILES: */C(C)=C/[C@@H](C)C(=O)c1ccc(N(C)C)cc1 ; */C(C)=C/[C@H](C)C(=O)c1ccc(N(C)C)cc1 ; */C=C/[C@@H](C)C(=O)c1ccc(N(C)C)cc1 ; */C=C/[C@H](C)C(=O)c1ccc(N(C)C)cc1]
or
selected from the group consisting of:

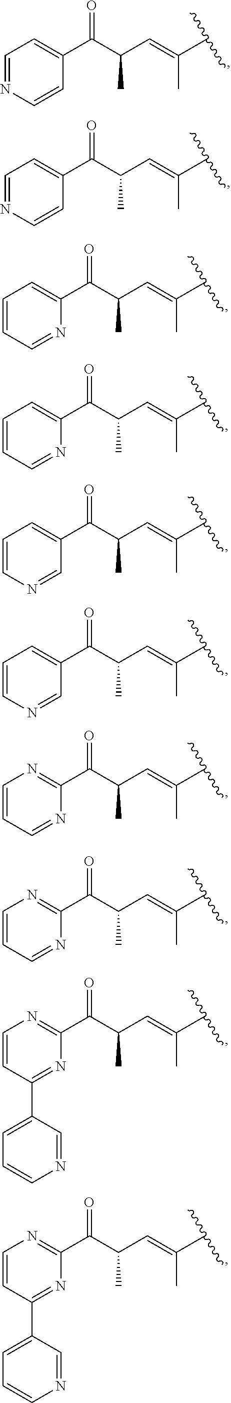

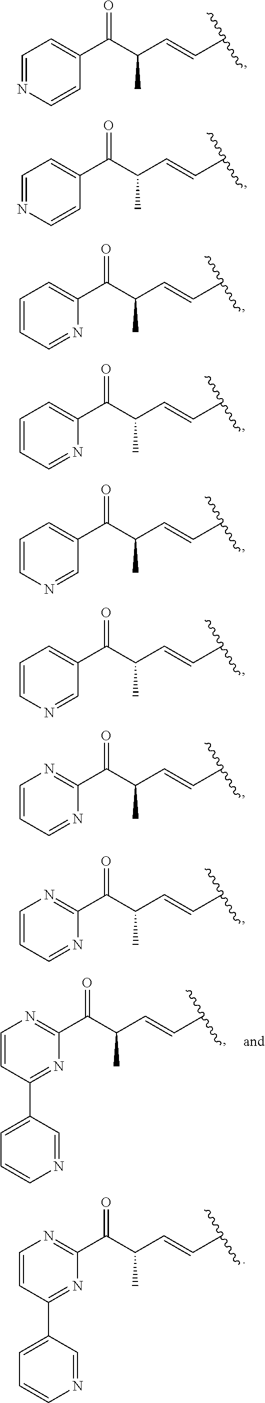

43. The method of claim 40, wherein R₁ is: 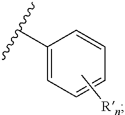
wherein
n is an integer between 0 and 5, inclusive; and
each occurrence of R′ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR^{B}; —C(═O)R^{B}; —CO₂R^{B}; C(═O)N(R^{B})₂; —CN; —SCN; —SR^{B}; —SOR^{B}; —SO₂R^{B}; —NO₂; —N(R^{B})₂; —NHC(O)R^{B}; or —C(R^{B})₃; wherein each occurrence of R_{B} is independently hydrogen; halogen; aliphatic; heteroaliphatic; acyl; aryl; heteroaryl; hydroxyl; alkoxy; aryloxy; alkylthioxy; arylthioxy; amino; alkylamino; dialkylamino; heteroaryloxy; or heteroarylthioxy.

44. The method of claim 43, wherein R′ is 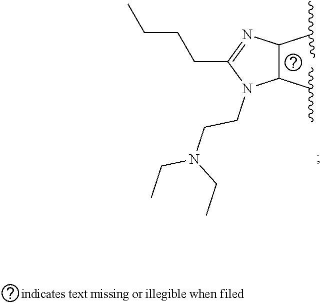 [SMILES: *C1N=C(CCCC)N(CCN(CC)CC)C1*]
or
wherein R₁ is selected from the group consisting of:

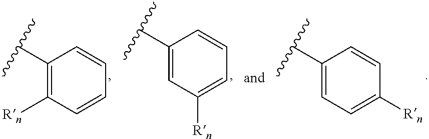

45. The method of claim 44, wherein R′ is selected from the group consisting of: 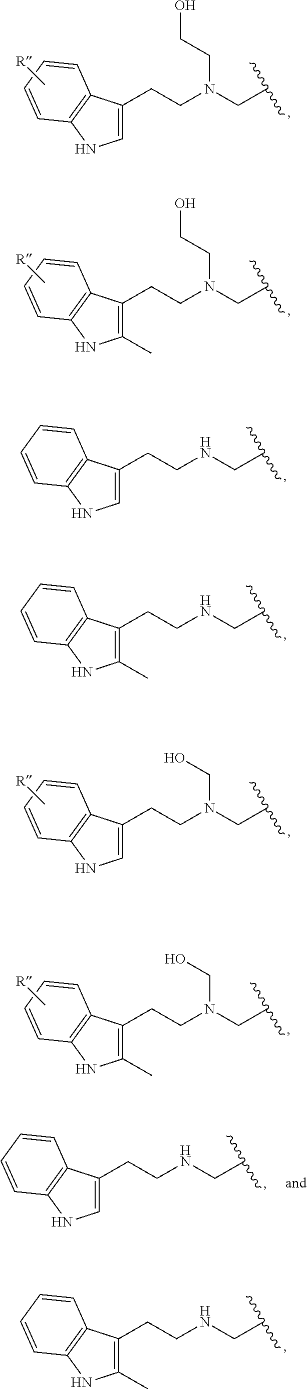
wherein each occurrence of R″ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR^{A}; —C(═O)R^{A}; —CO₂R^{A}; —C(═O)N(R^{A})₂; —CN; —SCN; —SR^{A}; —SOR^{A}; —SO₂R^{A}; —NO₂; —N(R^{A})₂; —NHC(O)R^{A}; or —C(R^{A})₃; wherein each occurrence of R^{A} is independently hydrogen; halogen; aliphatic; heteroaliphatic; acyl; aryl; heteroaryl; hydroxyl; alkoxy; aryloxy; alkylthioxy; arylthioxy; amino; alkylamino; dialkylamino; heteroaryloxy; or heteroarylthioxy.

46. The method of claim 44, wherein R′ is:
selected from the group consisting of: 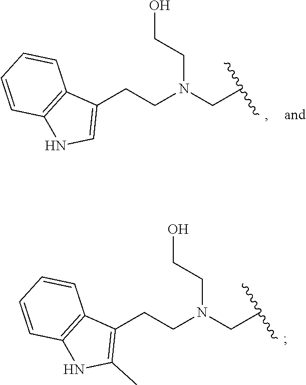 [SMILES: *CN(CCO)CCc1c[nH]c2ccccc12 ; *CN(CCO)CCc1c(C)[nH]c2ccccc12]
or
selected from the group consisting of: 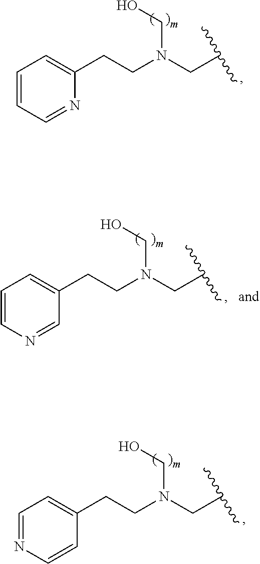
wherein m is an integer between 1 and 6, inclusive or
selected from the group consisting of: 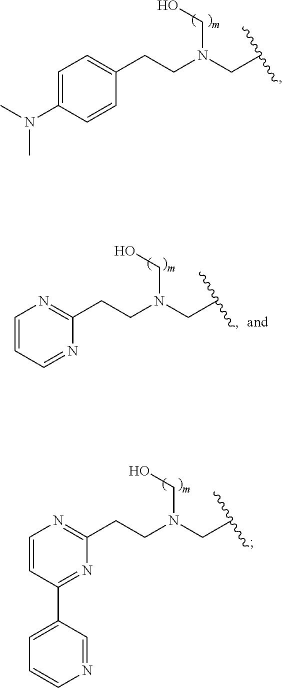
or
selected from the group consisting of:

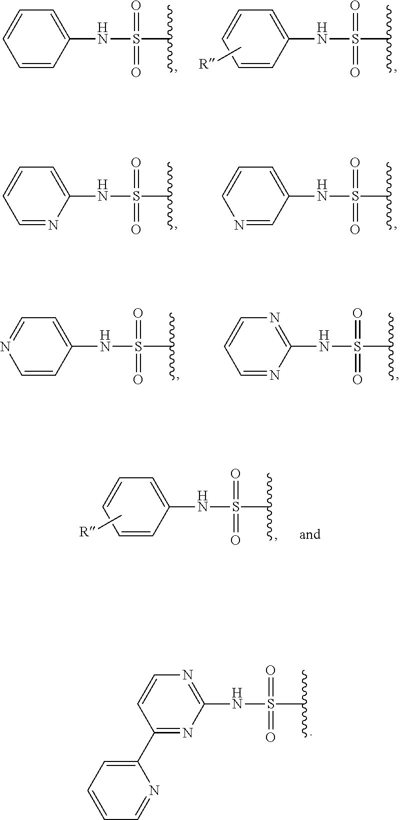

47. The method of claim 40, wherein R₁ is selected from the group consisting of:

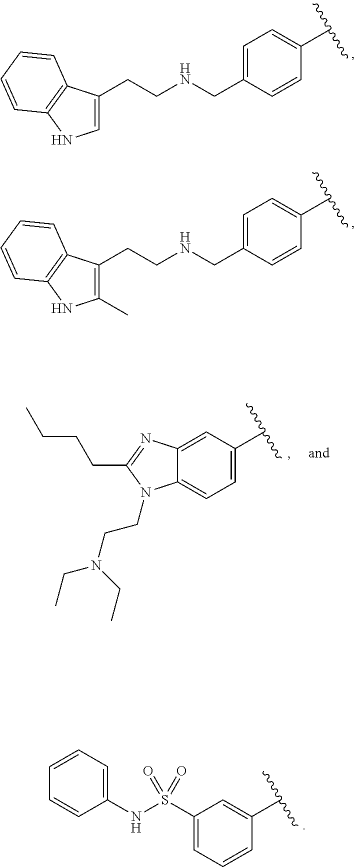 [SMILES: *c1ccc(CNCCc2c[nH]c3ccccc23)cc1 ; *c1ccc(CNCCc2c(C)[nH]c3ccccc23)cc1 ; *c1ccc2c(c1)nc(CCCC)n2CCN(CC)CC ; *c1cccc(S(=O)(=O)Nc2ccccc2)c1]

48. The method of claim 43, wherein R₁ is selected from the group consisting of: 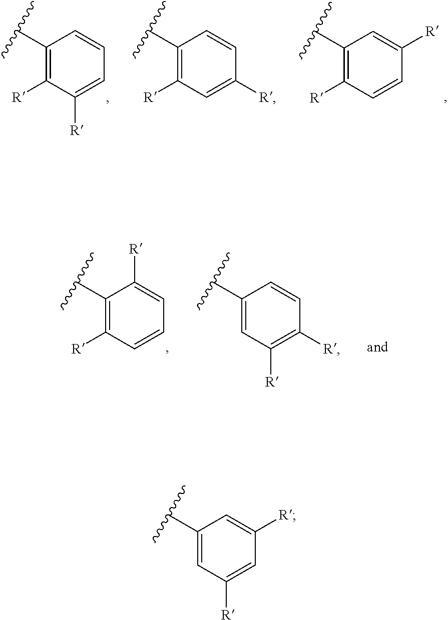
or
selected from the group consisting of:

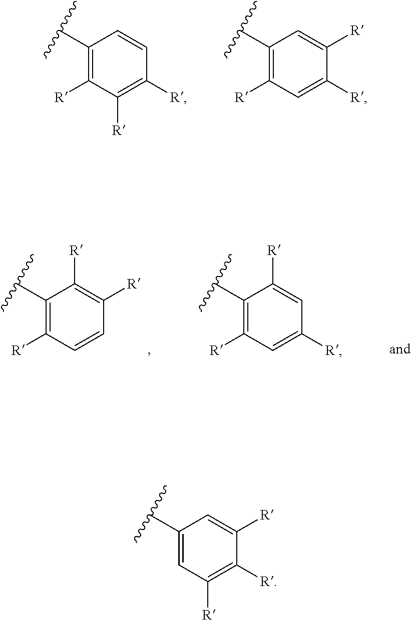

49. The method of claim 40, wherein R₁ is selected from the group consisting of:

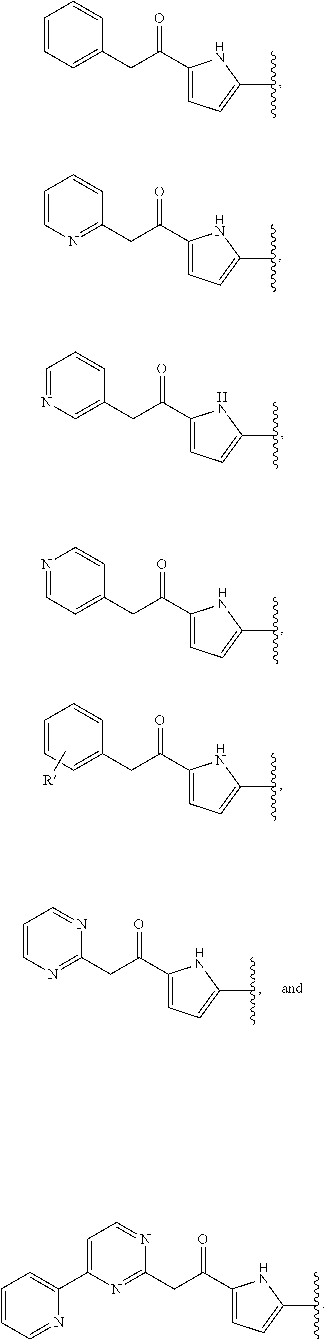

50. The method of claim 40, wherein R₁ is-C(O) R″, wherein R″ is hydrogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR^{B}; —N(R^{B})₂; —NHC(O)R^{B}; or —C(R^{B})₃; wherein each occurrence of R_{B} is independently hydrogen; halogen; aliphatic; heteroaliphatic; acyl; aryl; heteroaryl; hydroxyl; alkoxy; aryloxy; alkylthioxy; arylthioxy; amino; alkylamino; dialkylamino; heteroaryloxy; or heteroarylthioxy.

51. The method of claim 40, wherein R₁ is selected from a group consisting of: 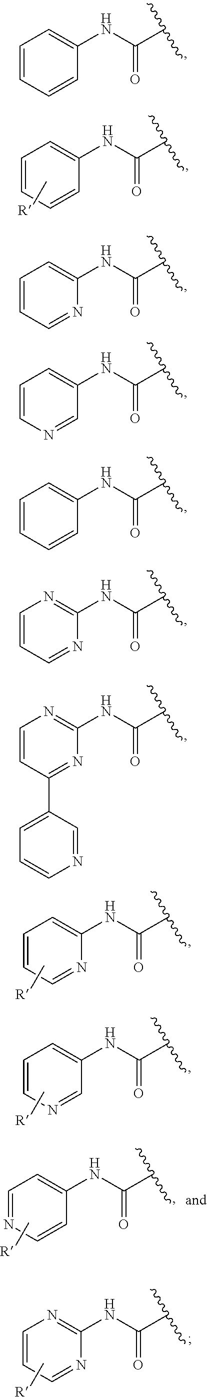
or
selected from the group consisting of: 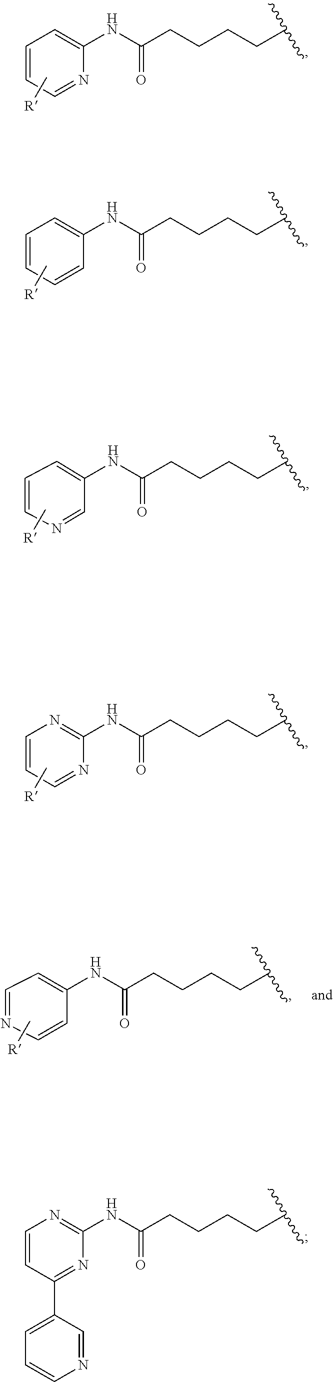
or
selected from the group consisting of: 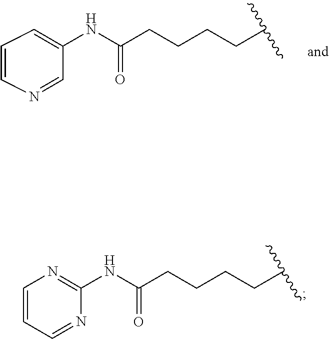 [SMILES: *CCCCC(=O)Nc1cccnc1 ; *CCCCC(=O)Nc1ncccn1]
or
selected from the group consisting of: 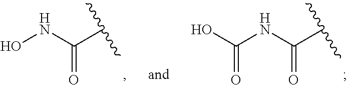 [SMILES: *C(=O)NO ; *C(=O)NC(=O)O]
or
R¹ is:

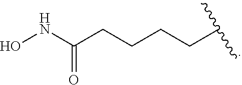 [SMILES: *CCCCC(=O)NO]

52. The method of claim 40, wherein R₁ is: 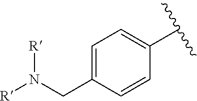
wherein each occurrence of R′ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR^{A}; —C(═O)R^{A}; —CO₂R^{A}; —C(═O)N(R^{A})₂; —CN; —SCN; —SR^{A}; —SOR^{A}; —SO₂R^{A}; —NO₂; —N(R^{A})₂; —NHC(O)R^{A}; or —C(R^{A})₃; wherein each occurrence of R^{A} is independently hydrogen; halogen; aliphatic; heteroaliphatic; acyl; aryl; heteroaryl; hydroxyl; alkoxy; aryloxy; alkylthioxy; arylthioxy; amino; alkylamino; dialkylamino; heteroaryloxy; or heteroarylthioxy.

53. The method of claim 40, wherein R₁ is selected from a group consisting of:

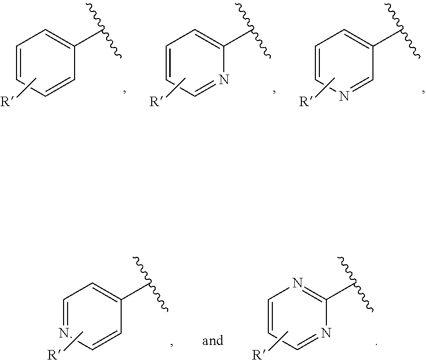

54. The method of claim 40, wherein R₁ is:

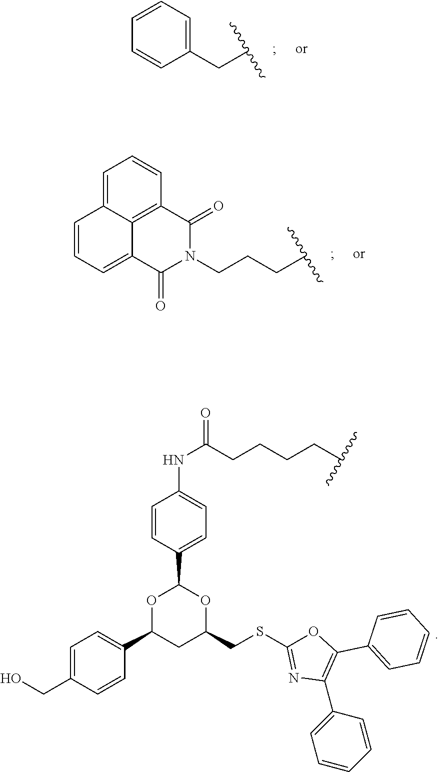 [SMILES: *Cc1ccccc1 ; *CCCN1C(=O)c2cccc3cccc(c23)C1=O ; *CCCCC(=O)Nc1ccc([C@H]2O[C@@H](CSc3nc(-c4ccccc4)c(-c4ccccc4)o3)C[C@@H](c3ccc(CO)cc3)O2)cc1]

55. The method of claim 40, wherein R₁ is: 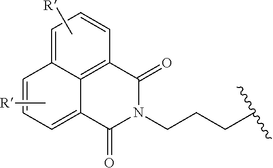
wherein R′ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR^{A}; —C(═O)R^{A}; —CO₂R^{A}; —C(═O)N(R^{A})₂; —CN; —SCN; —SR^{A}; —SOR^{A}; —SO₂R^{A}; —NO₂; —N(R^{A})₂; —NHC(O)R^{A}; or —C(R^{A})₃; wherein each occurrence of R^{A} is independently hydrogen; halogen; aliphatic; heteroaliphatic; acyl; aryl; heteroaryl; hydroxyl; alkoxy; aryloxy; alkylthioxy; arylthioxy; amino; alkylamino; dialkylamino; heteroaryloxy; or heteroarylthioxy.

56. The method of claim 40, wherein formula (I) has the structure

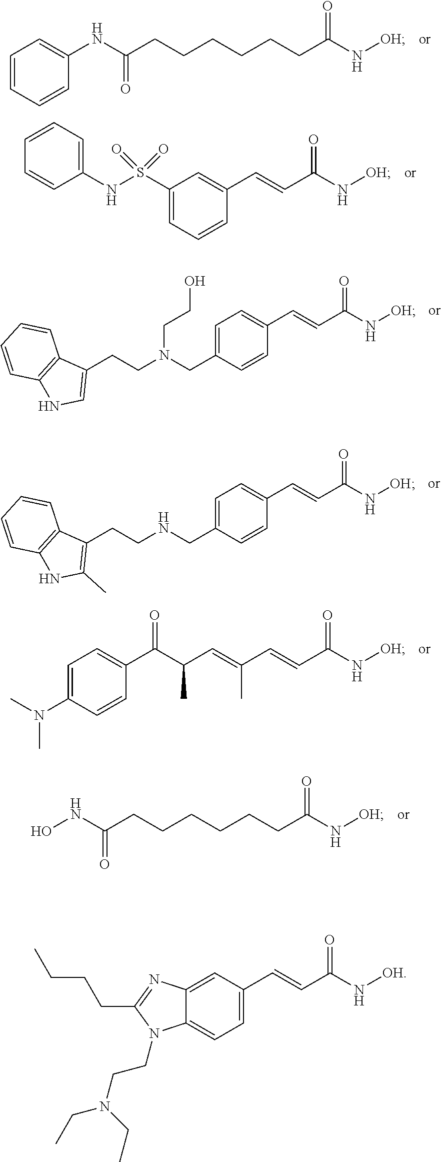

57. The method of claim 39, wherein the microorganism is a microalga, stramenophile, protist or fungus.

58. The method of claim 57, wherein the microalga is Chlorella vulgaris, Chlorella sorokiniana, Chlorella protothecoides, Chlamydomonas reinhardtii, Schizochytrium limacinum, Phaeodactylum tricornutum or Thalassiosira pseudonana.
